SEQUENCE LISTING (15 sequences)

SEQ ID NO: 1 - DNA, 9 bp, artificial sequence
  NRAS gene point mutation in AML patient-derived blasts
tgtccagct

SEQ ID NO: 2 - DNA, 9 bp, artificial sequence
  mutant NRAS gene
  misc_feature (2)..(2): n is a, c, g, t or u
tntccagct

SEQ ID NO: 3 - DNA, 10 bp, artificial sequence
  pyrosequencing at the corresponding positions
atgtgcagct

SEQ ID NO: 4 - protein, 16 aa, artificial sequence
  ACLY peptide-1 in the ATP-grasp domain
LVVKPDQLIKRRGKLG

SEQ ID NO: 5 - protein, 15 aa, artificial sequence
  ACLY peptide-2 in the CoA-binding domain
ALTRKLIKKADQKGV

SEQ ID NO: 6 - protein, 21 aa, artificial sequence
  tyrosine residues on ACLY
EGDYVLFHHEGGVDVGDVDAK

SEQ ID NO: 7 - protein, 10 aa, artificial sequence
  tyrosine residues on ACLY
VDATADYICK

SEQ ID NO: 8 - protein, 15 aa, artificial sequence
  tyrosine residues on ACLY
EAYPEEAYIADLDAK

SEQ ID NO: 9 - protein, 11 aa, artificial sequence
  tyrosine residues on ACLY
RGGPNYQEGLR

SEQ ID NO: 10 - protein, 12 aa, artificial sequence
  tyrosine residues on ACLY
LYRPGSVAYVSR

SEQ ID NO: 11 - protein, 15 aa, artificial sequence
  tyrosine residues on ACLY
TTDGVYEGVAIGGDR

SEQ ID NO: 12 - protein, 17 aa, artificial sequence
  tyrosine residues on ACLY
QHFPATPLLDYALEVEK

SEQ ID NO: 13 - protein, 1101 aa, homo sapien
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 14 - protein, 15 aa, artificial sequence
  ACLY peptide-2 in the CoA-binding domain (Kpeptide-2)
ALTRKLIAAADQKGV

SEQ ID NO: 15 - protein, 16 aa, artificial sequence
  ACLY peptide-1 in the grasp domain sequence
LVVKPDQLIKRRGKLG

(19) United States
(12) Patent Application Publication
(10) Pub. No.: US 2022/0218659 A1
(43) Pub. Date: Jul. 14, 2022

(54) PI3K/LYN-ACLY Signaling Inhibition

(71) Applicant: Institute For Cancer Research d/b/a The Research Institute of Fox Chase Cancer Center, Philadelphia, PA (US)

(72) Inventors: Andrzej Ptasznik, Philadelphia, PA (US); Johnvesly Basappa, Philadelphia, PA (US)

(21) Appl. No.: 17/613,267

(22) PCT Filed: May 26, 2020
(86) PCT No.: PCT/US20/34488

§ 371 Date: Nov. 22, 2021

Related U.S. Application Data
(60) Provisional application No. 62/852,991, filed on May 25, 2019.
(60) Provisional application No. 62/928,553, filed on Oct. 31, 2019.

(51) Int. Cl. A61K 31/365; A61K 31/20; A61K 31/18; A61K 45/06

(52) U.S. Cl. CPC A61K 31/365; A61K 45/06; A61K 31/18; A61K 31/20

(57) ABSTRACT

The present disclosure is directed, in part, to pharmaceutical compositions comprising a Src protein tyrosine kinase inhibitor, an ATP citrate lyase (ACLY) inhibitor, and a PI3K inhibitor, and/or a Src and PIP₂/PIP₃ inhibitor of binding to ACLY, and a pharmaceutically acceptable carrier, methods of identifying a compound as a potential therapeutic agent for treating a disease or condition associated with the ACLY/Acetyl-CoA metabolic pathway in a cell, and methods of treating a disease or condition associated with the ACLY/Acetyl-CoA metabolic pathway.

### FIELD

The present disclosure is directed, in part, to methods of identifying a compound as a potential therapeutic agent for treating a disease or condition associated with the ATP citrate lyase (ACLY)/Acetyl-CoA metabolic pathway.

### BACKGROUND

The most frequently activated signaling pathway in cancer is the phosphoinositide 3-kinase (PI3K) pathway (Traynor-Kaplan et al., Nature, 1988, 28, 353-356; Whitman et al., Nature, 1988, 14, 644-646; Goncalves et al., N. Engl. J. Med., 2018, 379, 2052-2062). This is principally due to at least one, but more often multiple, genetic modifications in PI3K/PTEN and/or upstream activators such as RAS subfamily proteins, receptor tyrosine kinases, and non-receptor tyrosine kinases including Src family kinases (SFK) that are common in all types of cancer (Goncalves et al., N. Engl. J. Med., 2018, 379, 2052-2062). Two key signaling molecules common to these pathways are the phospholipids, PI(4,5)P2 and PI(3,4,5)P3, whose alterations trigger cascades of pro-cancer responses such as cell proliferation, survival, adhesion and chemotaxis (Traynor-Kaplan et al., Nature, 1988, 28, 353-356; Whitman et al., Nature, 1988, 14, 644-646; Goncalves et al., N. Engl. J. Med., 2018, 379, 2052-2062). PI(4,5)P₂ and PI(3,4,5)P₃ couple to metabolic pathways through both Akt-dependent and Akt-independent mechanisms that can lead to tumor progression (Mahajan et al., J. Cell. Physiol., 2012, 227, 3178-3184). Src was the first transforming protein (Rous, J. Exp. Med., 1911, 13, 397-411) and protein tyrosine kinase (Hunter et al., Proc. Natl. Acad. Sci. USA, 1980, 77, 1311-1315) discovered. While the SFKs, particularly Lyn, are functionally and physically associated with PI3K (Ptasznik et al., J. Exp. Med., 2002, 196, 667-678), and constitutively activated in AML (Dos Santos et al., Blood, 2008, 111, 2269-2279), CMLblast crisis (Ptasznik et al., J. Exp. Med., 2002, 196, 667-678; Ptasznik et al., Nat. Med., 2004, 11, 1187-1189), breast cancer, glioblastoma and other hematologic and solid tumors, Lyn's peculiar role in cancer cell metabolism remains to be elucidated.

A fundamental feature of tumor progression is the reprogramming of metabolic pathways and gene regulation. ATP citrate lyase (ACLY) is a key enzyme that is a gatekeeper for the synthesis of Acetyl-CoA, a critical molecule delivering the acetyl groups for metabolism and gene regulation, i.e. biosynthesis of fatty acids/lipids and protein/histone acetylation, respectively (Zaidi et al., Cancer Res., 2012, 72, 3709-3714; Cai et al., Mol. Cell, 2011, 42, 426-437; Sivanand et al., Mol. Cell, 2017, 67, 252-265). ACLY, and the resulting lipid production and histone acetylation, are upregulated in cancer (Zaidi et al., Cancer Res., 2012, 72, 3709-3714; Cai et al., Mol. Cell, 2011, 42, 426-437).

### SUMMARY

The present disclosure provides pharmaceutical compositions comprising: a Src protein tyrosine kinase inhibitor, an ATP citrate lyase (ACLY) inhibitor, a PI3K inhibitor, and a pharmaceutically acceptable carrier.

The present disclosure also provides methods of identifying a compound as a potential therapeutic agent for treating a disease or condition associated with the ACLY/Acetyl-CoA metabolic pathway in a cell comprising: performing an assay to determine the ability of the compound to inhibit the interaction of PIP₂, PIP₃, and/or Lyn tyrosine kinase to ACLY, or the activity of a complex of PIP₂/Lyn tyrosine kinase/ACLY, or the activity of complex of PIP₃/Lyn tyrosine kinase/ACLY; wherein when the compound inhibits the interaction of PIP₂, PIP₃, and/or Lyn tyrosine kinase to ACLY, or inhibits the activity a complex of PIP₂/Lyn tyrosine kinase/ACLY, or inhibits the activity of a complex of PIP₃/Lyn tyrosine kinase/ACLY, the compound is a potential therapeutic agent.

The present disclosure also provides methods of treating a disease or condition associated with the ACLY/Acetyl-CoA metabolic pathway in a cell in a subject in need thereof comprising administering to the subject a Lyn tyrosine kinase inhibitor, an ACLY inhibitor, and a PI3K inhibitor to the subject.

The present disclosure also provides combinations of a Lyn tyrosine kinase inhibitor, an ACLY inhibitor, and a PI3K inhibitor for use in the manufacture of a medicament for treating a disease or condition associated with the ACLY/Acetyl-CoA metabolic pathway in a cell.

The present disclosure also provides uses of a pharmaceutical composition comprising a Lyn tyrosine kinase inhibitor, an ACLY inhibitor, and a PI3K inhibitor for treating a disease or condition associated with the ACLY/Acetyl-CoA metabolic pathway in a cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows that PI(4,5)P₂ and PI(3,4,5)P₃ directly interact with AC LY in acute myeloid leukemia (AML) patient-derived marrow blasts, but not non-malignant marrow CD34+ cells; treatment of AML, and normal cells with the tri-functional membrane-permeant PIP₂ or PIP₃ derivatives; the chemical structure of trifunctional PI(4,5)P₂ is shown.

FIG. 1B shows normalized reporter ion intensities from +UV samples were divided by the respective −UV control values to yield the final enrichment factor; the bars show the average enrichment ratio (+UV/−UV) for each condition, the p values show the relation between +UV and −UV samples in each condition and they are highly statistically significant; ACLY was enriched with PIP₂ or PIP₃ more than 100% in AML patient cells while no enrichment was obtained in illuminated normal cells.

FIG. 1C shows that ACLY is present in PI(4,5)P₂ precipitates from acute myeloid leukemia HL-60 cells; cells were lysed and immunoprecipitated with anti-PIP₂ or IgG control and blotted for ACLY; the input (5%) lysate was also analyzed; the position of ACLY is indicated; binding of negatively charged PIP₂ to positively charged amino acids on ACLY (co-immunoprecipitate) can cause slight change in electrophoretic mobilities in SDS-PAGE gel, as compared to input.

FIG. 1D shows colocalization of ACLY and PI(4,5)P₂ in HL-60 cells; immunoreactivity for ACLY is shown in red and PIP₂ in green; when the two fluorescence spectra are merged (right panel) ACLY and PIP₂ colocalization in the cell is shown by the yellow color; confocal imaging with 60× magnification (data not shown) indicates PIP₂ and ACLY colocalization throughout the cell, particularly in the cell membranes; the colocalization of ACLY and PI(3,4,5)P₃ was also measured, but levels of endogenous PIP₃ were too low in these cells to be analyzed by this method.

FIG. 2A shows that ACLY selectively interacts with the phosphorylated phosphoinositides (PIP, PIP₂, PIP₃), Phosphatidic Acid and Phosphatidylserine, but not with phosphatidylinositol (PI) and nine other lipids.

FIG. 2B shows membrane dots densitometry values were measured and used for the graph.

FIG. 2C shows the ACLY peptide-2 (Co-A-binding domain), but not the ACLY peptide-1 (ATP-binding domain), binds to PI(4,5)P₂; schematic representation of a PIP specificity plate.

FIG. 2D shows 96-well polystyrene microplate where each row has an individual phosphoinositide coated at 20 pmols per well; the two ACLY peptides were designed, synthetized and used to detect phospholipids binding on the PIP specificity plates.

FIG. 2E shows ACLY peptides 1 & 2 binding; for statistical analysis Graphpad prism software was used; error bars, S.E.; n=3, one-way ANOVA or unpaired t-test; asterisks indicate significant difference between PI(4,5)P₂ and other phospholipids. ***, p<0.0001.

FIG. 2F shows binding of the ACLY peptide-2 to PIP₂ is decreased in the presence of Coenzyme A (PIP₂:CoA) or with mutant ACLY peptide-2 with replacement of basic amino acid lysine(K) to alanine (A) (PIP₂:K-A); for ACLY and PIP₂ specificity binding assay was performed using the N-terminally FITC labelled wild type ACLY peptide-2 (CoA-binding domain sequence-peptide-2: ALTRKLIKKADQKGV; SEQ ID NO:5), or in ACLY peptide-2 two basic amino acids lysine (K) were replaced with alanine (Kpeptide-2: ALTRKLIAAADQK GV; SEQ ID NO:14), with or without 50 μM CoA in binding conditions; for statistical analysis Graphpad prism software was used; error bars, S.E.; n=3, one-way ANOVA or unpaired t-test; asterisks indicate significant difference from PIP₂ to other conditions; ***, p<0.0001.

FIG. 2G shows the ACLY full length protein binds to PIP₂ in Src protein tyrosine kinase-dependent and PI 3-kinase-dependent manner; HEK293T cells were transfected with full length ACLY-HA alone or in co-transfection of active SRC kinase; after 36 hours of transfection cells were subjected to DMSO or Dasatinib (2 μM) or BKM120 (2 μM) for 15 hours and lysed in cell lysis buffer and followed the PIP₂ immunoprecipitation and western blotting; the phospho-ACLY bound to PIP₂ were quantified using IMAGE software to analyze densitometry values for quantitation using PRISM Graphpad statistical analysis tool; asterisks indicate significant difference from DMSO to Dasatinib (Src inhibitor) or BKM120 (PI3K inhibitor); **, p<0.001.

FIG. 3A shows Lyn directly interacts and phosphorylates the tyrosine residues of ACLY; Src family kinase phosphorylate ACLY on the tyrosine residues; ACLY-HA and ACLY-HA+SRC transfected human HEK293T cells were lysed, precipitated with HA or IgG control antibody and blotted for p-ACLY (pan Tyrosine Y100), p-SRC (Y416) and HA; cells transfected with Src showed remarkable induction of ACLY tyrosine phosphorylation and phosphorylated SRC (Y416) was present in ACLY-HA precipitates; the input lysate was also analyzed; results are representative of two independent experiments.

FIG. 3B shows LYN directly phosphorylates ACLY on the tyrosine residue; in vitro tyrosine kinase assay on ACLY (left panel): recombinant HA-tagged ACLY (non-phosphorylated form) was purified and was incubated with immunoprecipitated Lyn (from total lysates of HL-60 AML cells treated with DMSO or 500 nM Bafetinib for 16 hours) in an in vitro kinase assay buffer and subsequently blotted with the indicated antibodies; the ACLY protein is phosphorylated on tyrosine residue only in the presence of active LYN (pY396) and this tyrosine phosphorylation of ACLY is prevented by the Lyn kinase inhibitor, Bafetinib; as indicated in the right panel, ACLY is present in Lyn immunoprecipitates in HL-60 AML cells (5% input)

FIGS. 3C and 3D show phosphoproteomics analysis of ACLY in vitro phosphorylated samples; active recombinant Lyn or Src kinase directly phosphorylated purified His-tagged ACLY at tyrosine residues in an in vitro kinase assay; phosphorylated Lyn (Y396) and Src (Y416) and also ACLY were detected by pan phospho-Tyrosine antibody (pY100); in vitro tyrosine phosphorylated ACLY samples were resolved on 10% Novex gels and stained with colloidal blue (see, FIG. 3D); the bands were excised and samples were evaluated by phosphoproteomics analysis.

FIG. 3E shows that phosphoproteomics analysis resulted in identification of novel Lyn kinase or Src kinase mediated tyrosine phosphorylation sites of ACLY; sites on ACLY which are common in both Lyn and Src are highlighted in red.

FIG. 4A shows PI3K and Lyn inhibitors suppress the ACLY enzyme activity, synthesis of Acetyl-CoA and Acetyl-CoA-dependent downstream activities (histone acetylation, cell growth) in AML cells; effect of PI3K and Lyn inhibitors on the ACLY enzyme activity and synthesis of Acetyl-CoA; the ACLY enzyme activity assay on HL-60 cells treated with DMSO, LYN kinase inhibitor, Bafetinib (1.0 μM) or PI3Kinase inhibitor BKM120 (2.0 μM) or AKT inhibitor, Capivasertib (5 μM) for 16 hours and lysates; error bars, S.E.; n=3, one-way ANOVA analysis; asterisks indicate significant difference from DMSO/Capivasetib to Bafetinib/BKM120; ***, p<0.0001.

FIG. 4B shows HL-60 cells were treated with Lyn inhibitor (Bafetinib) or PI3K inhibitors (LY204002, BKM120) or vehicle for 16 hours for Acetyl-CoA measurement; control values were the means of 3 DMSO control samples; Student's unpaired t-test was used to compare the DMSO control vs inhibitor treatment group; data are shown as mean±SEM; ***p<0.0001, n=3/FIGS. 4C, 4D, and 4E show effect of Lyn, PI3K and ACLY (BMS303141) inhibitors on AML cell growth; HL-60 cells were treated with various concentrations of the inhibitors or vehicle (0.1% DMSO) in the presence of 10% FBS in RPMI media for 72 hours for MTT assay; error bars, S.E.; n=3, one-way ANOVA analysis; asterisks indicate significant difference from DMSO to Bafetinib or BKM120 or BMS303141; ***, p<0.0001.

FIG. 4F shows effect of Lyn and PI3K inhibitors on Histone H3 acetylation; HL-60 cells were treated with the Lyn inhibitor or PI3K inhibitors or vehicle in the presence of 10% FBS in RPMI for 16 hours and then were blotted for H3K9ac, p-SRC Y416 (p-LYN Y396) and p-ACLY S454; densitometric analysis showed that Histone H3 acetylation was effectively blocked by the treatment of cells with Lyn inhibitor Bafetinib (90%) and PI3K inhibitors, LY294004 (60%) or BKM120 (97%); the treatment with inhibitors did not suppress serine-threonine phosphorylation of ACLY (p-ACLY S454), which is an AKT-mediated event; the pan-PI3K inhibitors at higher concentrations (2.5 μM LY294002 or 500 nM BKM120) also partially suppressed the Lyn activity (˜40-50%), since Lyn is coupled to PI3K in HL-60 cells and Src family kinases can be phosphorylated by PI3K.

FIG. 5 shows the effect of Lyn and PI3K inhibitors on Fatty Acid composition of PI, PIP, and PIP₂; HL-60 cells were treated with Lyn inhibitor (Bafetinib, BAF) or PI3K inhibitors (BKM120, BKM or LY294002, LY) or vehicle in the presence of 10% FBS in RPMI for 16 hours for lipidomic analysis; the treatment with the inhibitors resulted in an overall decrease in levels of total PI/PIP/PIP₂ (as compared to DMSO control—100%) and the species of PIs with shorter fatty acid chains (32:0, 34:0) were most affected by the inhibitors, in a manner consistent with ACLY inhibition; control values were the means of 3 DMSO control samples against which values from individual treated samples were calculated; data are means±SD, n=3.

FIG. 6A shows a schematic presentation of the ACLY PIP₂ binding region and the novel Lyn/Src-dependent tyrosine phosphorylation sites; the three tyrosine phosphorylation sites identified in these experiments described herein and common in Lyn/Src kinase mediated ACLY phosphorylation are shown, including Y682 (catalytic domain), Y252 (citrate-binding domain) and Y227 (ATP-binding domain); the ACLY peptides which were used in these experiments are shown (peptide-1 in the ATP-binding domain sequence and peptide-2 in the CoA-binding domain sequence); the PIP₂ binding motif on ACLY, which was detected using the ACLY peptide-2, is shown.

FIG. 6B shows a proposed model for interaction between oncogenic signal transduction pathways and Acetyl-CoA metabolic pathway in transformed cells; PI3K and Src family kinases-mediated pathways are the most frequently activated signaling pathways in cancer; as indicated on the right, it is proposed that Lyn/Src oncogenic kinase-mediated tyrosine phosphorylation of ACLY induces its interaction with phospholipids where ACLY directly binds to PI(4,5)P₂ and other phospholipids in cancer cells; these interactions lead to increased ACLY-dependent Acetyl-CoA synthesis, which may in turn lead to the increase of phospholipid synthesis (including PIP₂) and protein acetylation in cancer cells; the basis for a persistent interaction of PIP₂/PIP₃ with ACLY remains to be defined, but it could result from Lyn/Src-mediated phosphorylation of ACLY and/or increased Lyn/PI3K-mediated PIP₂ synthesis and/or direct oncogene-mediated alteration of PIP₂ in the cell membrane and/or the nuclear compartment of cancer cells; thus, it is proposed a Src family tyrosine kinase and PI3K-dependent mechanism whereby oncoproteins hijack a major, Acetyl-CoA-mediated, metabolic pathway fueling synthesis of phospholipids and growth of cancer cells; this paradigm may provide further insight into the striking ability of PI3K and Src kinases to transform various cell types (red color on the right stands for the oncogenic constitutive activation pathways in cancer cells; RTKs on the left in normal cells:Receptor Tyrosine Kinases, the dashed line represents a regulatory temporary stimulation via cytokine/RTK and other receptors in normal cells).

FIG. 7 shows an analysis of NRAS gene Q61K point mutation in ML patient-derived blasts; gDNA was analyzed by pyrosequencing.

FIG. 8 shows AML samples collected from the patient and his normal cells were analyzed by the whole-exome sequencing (WES); the listed mutations reflect tumor-specific mutations with high allelic frequency (≥40%); the evaluation based on the predictive pathogenic algorithms, COSMIC and other databases.

FIG. 9 shows a comparison of effects of Lyn and PI3K inhibition on levels of select species of PI, PIP, and PIP₂ in HL60 cells determined by LC/MS/MS using Waters Xevo TQ MS/MS in MRM mode; data are means and standard deviations of Peak areas normalized to internal standard and protein (n=3): ng/mg protein (t-test, P values).

FIG. 10A shows human ACLY protein sequence; the ACLY protein sequences highlighted tyrosine phosphorylated sites are in red (common for Lyn and Src), blue (only Lyn), green (only Src) and the tested binding domains are highlighted in yellow; underlining indicates the ACLY region used to synthesis N-terminally biotin tagged synthetic peptides, as probes for ACLY-PIP specificity binding studies.

FIG. 10B shows Y682 is a highly conserved tyrosine residue, which means that has been maintained by natural selection.

### DESCRIPTION OF EMBODIMENTS

The present disclosure describes signaling and metabolic consequences in the ACLY/Acetyl-CoA metabolic pathway of multiple pathogenic chromosomal aberrations and genetic mutations by measuring binding of PIP₂ and/or PIP₃ to ACLY in AML patient-derived and normal donor-derived marrow cells. In the present disclosure, the effects of PI3K and Lyn inhibition on the Acetyl-CoA and fatty acid/phospholipid synthesis, histone acetylation, and growth of HL-60 AML cells is described. In addition, the present disclosure provides a novel mechanism in which the substrate and product of PI3K activity, PIP₂ and/or PIP₃, respectively, bind to Lyn-phosphorylated ACLY and couple oncogenic signaling events to the Acetyl-CoA synthesis and phospholipid metabolism and histone acetylation in AML cells.

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the term “carrier” means a diluent, adjuvant, or excipient with which a compound is administered in a composition.

As used herein, the term, “compound” means all stereoisomers, tautomers, isotopes, and polymorphs of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the terms “individual,” “subject,” and “patient,” used interchangeably, mean any animal described herein.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The present disclosure provides compositions, such as pharmaceutical compositions, comprising: one or more Src protein tyrosine kinase inhibitors, one or more ATP citrate lyase (ACLY) inhibitors, and one or more PI3K inhibitors, and one or more carriers, such as pharmaceutically acceptable carriers. In some embodiments, the pharmaceutical compositions, comprise: a Src protein tyrosine kinase inhibitor, an ACLY inhibitor, and a PI3K inhibitor, and a pharmaceutically acceptable carrier.

In some embodiments, the one or more ACLY inhibitors is BMS303141, MEDICA16, SB204990, or NDI-091143, or any combination thereof. In some embodiments, the ACLY inhibitor is BMS303141. In some embodiments, the ACLY inhibitor is MEDICA16. In some embodiments, the ACLY inhibitor is SB204990. In some embodiments, the ACLY inhibitor is NDI-091143.

In some embodiments, the ACLY inhibitor(s) is present in the composition in an amount from about 1 mg to about 500 mg, from about 50 mg to about 400 mg, from about 75 mg to about 300 mg, or from about 100 mg to about 200 mg. In some embodiments, the ACLY inhibitor is present in an amount from about 1 mg to about 500 mg. In some embodiments, the ACLY inhibitor is present in an amount from about 50 mg to about 400 mg. In some embodiments, the ACLY inhibitor is present in an amount from about 75 mg to about 300 mg. In some embodiments, the ACLY inhibitor is present in an amount from about 100 mg to about 200 mg. In some embodiments, the ACLY inhibitor is present in the composition in an amount from about 1 mg to about 50 mg, from about 1 mg to about 40 mg, from about 1 mg to about 30 mg, from about 1 mg to about 20 mg, or from about 1 mg to about 10 mg. In some embodiments, the ACLY inhibitor is present in an amount from about 1 mg to about 50 mg. In some embodiments, the ACLY inhibitor is present in an amount from about 1 mg to about 40 mg. In some embodiments, the ACLY inhibitor is present in an amount from about 1 mg to about 30 mg. In some embodiments, the ACLY inhibitor is present in an amount from about 1 mg to about 20 mg. In some embodiments, the ACLY inhibitor is present in an amount from about 1 mg to about 10 mg.

In some embodiments, the one or more PI3K inhibitors is LY294002, BKM120, voxtalisib, umbralisib, copanlisib, duvelisib, or alpelisib, or any combination thereof. In some embodiments, the PI3K inhibitor is LY294002. In some embodiments, the PI3K inhibitor is BKM120. In some embodiments, the PI3K inhibitor is voxtalisib. In some embodiments, the PI3K inhibitor is umbralisib. In some embodiments, the PI3K inhibitor is copanlisib. In some embodiments, the PI3K inhibitor is duvelisib. In some embodiments, the PI3K inhibitor is alpelisib.

In some embodiments, the PI3K inhibitor(s) is present in the composition in an amount from about 1 mg to about 500 mg, from about 50 mg to about 400 mg, from about 75 mg to about 300 mg, or from about 100 mg to about 200 mg. In some embodiments, the PI3K inhibitor is present in an amount from about 1 mg to about 500 mg. In some embodiments, the PI3K inhibitor is present in an amount from about 50 mg to about 400 mg. In some embodiments, the PI3K inhibitor is present in an amount from about 75 mg to about 300 mg. In some embodiments, the PI3K inhibitor is present in an amount from about 100 mg to about 200 mg. In some embodiments, the PI3K inhibitor is present in the composition in an amount from about 1 mg to about 50 mg, from about 1 mg to about 40 mg, from about 1 mg to about 30 mg, from about 1 mg to about 20 mg, or from about 1 mg to about 10 mg. In some embodiments, the PI3K inhibitor is present in an amount from about 1 mg to about 50 mg. In some embodiments, the PI3K inhibitor is present in an amount from about 1 mg to about 40 mg. In some embodiments, the PI3K inhibitor is present in an amount from about 1 mg to about 30 mg. In some embodiments, the PI3K inhibitor is present in an amount from about 1 mg to about 20 mg. In some embodiments, the PI3K inhibitor is present in an amount from about 1 mg to about 10 mg.

In some embodiments, the one or more Src protein tyrosine kinase inhibitors is a Lyn tyrosine kinase inhibitor. In some embodiments, the Lyn tyrosine kinase inhibitor(s) is bafetinib, bosutinib, masitinib, soracatinib, AZ 628, TC-S 7003, or PRT 062607, or any combination thereof. In some embodiments, the Lyn tyrosine kinase inhibitor is bafetinib. In some embodiments, the Lyn tyrosine kinase inhibitor is bosutinib. In some embodiments, the Lyn tyrosine kinase inhibitor is masitinib. In some embodiments, the Lyn tyrosine kinase inhibitor is soracatinib. In some embodiments, the Lyn tyrosine kinase inhibitor is AZ 628. In some embodiments, the Lyn tyrosine kinase inhibitor is TC-S 7003. In some embodiments, the Lyn tyrosine kinase inhibitor is PRT 062607.

In some embodiments, the Lyn tyrosine kinase inhibitor is present in the composition in amount from about 1 mg to about 100 mg, from about 5 mg to about 75 mg, from about 10 mg to about 60 mg, or from about 12.5 mg to about 50 mg. In some embodiments, the Lyn tyrosine kinase inhibitor is present in amount from about 1 mg to about 100 mg. In some embodiments, the Lyn tyrosine kinase inhibitor is present in amount from about 5 mg to about 75 mg. In some embodiments, the Lyn tyrosine kinase inhibitor is present in amount from about 10 mg to about 60 mg. In some embodiments, the Lyn tyrosine kinase inhibitor is present in amount from about 12.5 mg to about 50 mg. In some embodiments, the Lyn tyrosine kinase inhibitor is present in the composition in amount from about 15 mg to about 40 mg, from about 20 mg to about 35 mg, or from about 25 mg to about 30 mg. In some embodiments, the Lyn tyrosine kinase inhibitor is present in amount from about 15 mg to about 40 mg. In some embodiments, the Lyn tyrosine kinase inhibitor is present in amount from about 20 mg to about 35 mg. In some embodiments, the Lyn tyrosine kinase inhibitor is present in amount from about 25 mg to about 30 mg.

In some embodiments, the pharmaceutical composition is an oral dosage formulation, an intravenous dosage formulation, a topical dosage formulation, an intraperitoneal dosage formulation, or an intrathecal dosage formulation. In some embodiments, the pharmaceutical composition is an oral dosage formulation. In some embodiments, the pharmaceutical composition is an intravenous dosage formulation. In some embodiments, the pharmaceutical composition is a topical dosage formulation. In some embodiments, the pharmaceutical composition is an intraperitoneal dosage formulation. In some embodiments, the pharmaceutical composition is an intrathecal dosage formulation.

In some embodiments, the oral dosage formulation is a pill, tablet, capsule, cachet, gel-cap, pellet, powder, granule, or liquid. In some embodiments, the oral dosage formulation is a pill. In some embodiments, the oral dosage formulation is a tablet. In some embodiments, the oral dosage formulation is a capsule. In some embodiments, the oral dosage formulation is a gel-cap. In some embodiments, the oral dosage formulation is a liquid.

In some embodiments, the oral dosage formulation is protected from light and present within a blister pack or bottle. In some embodiments, the oral dosage formulation is within a blister pack. In some embodiments, the oral dosage formulation is a capsule. In some embodiments, the capsule comprises about 12.5 mg, about 25 mg, about 37.5 mg, or about 50 mg of the Lyn tyrosine kinase inhibitor. In some embodiments, the capsule comprises about 12.5 mg of the Lyn tyrosine kinase inhibitor. In some embodiments, the capsule comprises about 25 mg of the Lyn tyrosine kinase inhibitor. In some embodiments, the capsule comprises about 37.5 mg of the Lyn tyrosine kinase inhibitor. In some embodiments, the capsule comprises about 50 mg of the Lyn tyrosine kinase inhibitor. In some embodiments, the capsule comprises about 25 mg, about 50 mg, about 75 mg, about 100 mg, about 150 mg, or about 200 mg of the ACLY inhibitor. In some embodiments, the capsule comprises about 25 mg of the ACLY inhibitor. In some embodiments, the capsule comprises about 50 mg of the ACLY inhibitor. In some embodiments, the capsule comprises about 75 mg of the ACLY inhibitor. In some embodiments, the capsule comprises about 100 mg of the ACLY inhibitor. In some embodiments, the capsule comprises about 150 mg of the ACLY inhibitor. In some embodiments, the capsule comprises about 200 mg of the ACLY inhibitor. In some embodiments, the capsule comprises about 25 mg, about 50 mg, about 75 mg, about 100 mg, about 150 mg, or about 200 mg of the PI3K inhibitor. In some embodiments, the capsule comprises about 25 mg of the PI3K inhibitor. In some embodiments, the capsule comprises about 50 mg of the PI3K inhibitor. In some embodiments, the capsule comprises about 75 mg of the PI3K inhibitor. In some embodiments, the capsule comprises about 100 mg of the PI3K inhibitor. In some embodiments, the capsule comprises about 150 mg of the PI3K inhibitor. In some embodiments, the capsule comprises about 200 mg of the PI3K inhibitor.

In some embodiments, the intravenous dosage formulation is within an intravenous bag.

The present disclosure also provides methods of identifying one or more compounds as a potential therapeutic agent(s) for treating a disease or condition associated with the ACLY/Acetyl-CoA metabolic pathway in a cell. In some embodiments, the methods comprise performing an assay to determine the ability of the compound to inhibit the interaction of PIP₂, PIP₃, and/or Lyn tyrosine kinase to ACLY, or the activity of a complex of PIP₂/Lyn tyrosine kinase/ACLY, or the activity of complex of PIP₃/Lyn tyrosine kinase/ACLY.

In some embodiments, the methods comprise performing an assay to determine the ability of the compound to inhibit the interaction of PIP₂ and/or Lyn tyrosine kinase to ACLY. In some embodiments, the methods comprise performing an assay to determine the ability of the compound to inhibit the interaction of PIP₂ and Lyn tyrosine kinase to ACLY. In some embodiments, the methods comprise performing an assay to determine the ability of the compound to inhibit the interaction of PIP₂ or Lyn tyrosine kinase to ACLY.

In some embodiments, the methods comprise performing an assay to determine the ability of the compound to inhibit the interaction of PIP₃ and/or Lyn tyrosine kinase to ACLY. In some embodiments, the methods comprise performing an assay to determine the ability of the compound to inhibit the interaction of PIP₃ and Lyn tyrosine kinase to ACLY. In some embodiments, the methods comprise performing an assay to determine the ability of the compound to inhibit the interaction of PIP₃ or Lyn tyrosine kinase to ACLY.

In some embodiments, the methods comprise performing an assay to determine the ability of the compound to inhibit the interaction of PIP₂ and PIP₃ and/or Lyn tyrosine kinase to ACLY. In some embodiments, the methods comprise performing an assay to determine the ability of the compound to inhibit the interaction of PIP₂ and PIP₃ and Lyn tyrosine kinase to ACLY. In some embodiments, the methods comprise performing an assay to determine the ability of the compound to inhibit the interaction of PIP₂ and PIP₃ or Lyn tyrosine kinase to ACLY.

In some embodiments, the methods comprise performing an assay to determine the ability of the compound to inhibit the activity of a complex of PIP₂/Lyn tyrosine kinase/ACLY. In some embodiments, the methods comprise performing an assay to determine the ability of the compound to inhibit the activity of a complex of PIP₃/Lyn tyrosine kinase/ACLY. In some embodiments, the methods comprise performing an assay to determine the ability of the compound to inhibit the activity of a complex of PIP₂/PIP₃/Lyn tyrosine kinase/ACLY. The inhibition of the activity of the particular complex need not be complete inhibition. In some embodiments, the inhibition of activity of the complex is at least 10% inhibition. In some embodiments, the inhibition of activity of the complex is at least 20% inhibition. In some embodiments, the inhibition of activity of the complex is at least 30% inhibition. In some embodiments, the inhibition of activity of the complex is at least 40% inhibition. In some embodiments, the inhibition of activity of the complex is at least 50% inhibition. In some embodiments, the inhibition of activity of the complex is at least 60% inhibition. In some embodiments, the inhibition of activity of the complex is at least 70% inhibition. In some embodiments, the inhibition of activity of the complex is at least 80% inhibition. In some embodiments, the inhibition of activity of the complex is at least 90% inhibition.

In some embodiments, when the compound inhibits the interaction of PIP₂ and/or Lyn tyrosine kinase to ACLY, inhibits the interaction of PIP₂ and Lyn tyrosine kinase to ACLY, inhibits the interaction of PIP₂ or Lyn tyrosine kinase to ACLY, inhibits the interaction of PIP₃ and/or Lyn tyrosine kinase to ACLY, inhibits the interaction of PIP₃ and Lyn tyrosine kinase to ACLY, inhibits the interaction of PIP₃ or Lyn tyrosine kinase to ACLY, inhibits the interaction of PIP₂ and PIP₃ and/or Lyn tyrosine kinase to ACLY, inhibits the interaction of PIP₂ and PIP₃ and Lyn tyrosine kinase to ACLY, or inhibits the interaction of PIP₂ and PIP₃ or Lyn tyrosine kinase to ACLY, the compound is a potential therapeutic agent.

In some embodiments, when the compound inhibits the activity of a complex of PIP₂/Lyn tyrosine kinase/ACLY, inhibits the activity of a complex of PIP₃/Lyn tyrosine kinase/ACLY, or inhibits the activity of a complex of PIP₂/PIP₃/Lyn tyrosine kinase/ACLY, the compound is a potential therapeutic agent.

In some embodiments, the compound is any potential therapeutic agent such as, for example, a small molecule, an antibody, a nucleic acid molecule, a peptide, or a protein. In some embodiments, the compound is a small molecule. In some embodiments, the compound is an antibody. In some embodiments, the compound is a nucleic acid molecule. In some embodiments, the compound is a peptide. In some embodiments, the compound is a protein. In some embodiments, the peptide is a cell permeable synthetic peptide, which can be used to prevent the effect of PIP₂ and PIP₃ on ACLY or the effect of Lyn on ACLY. In some embodiments, the antibody can be a monoclonal antibody blocking phospho-ACLY. In some embodiments, the nucleic acid molecule can be a miRNA or siRNA or antisense oligonucleotide.

In some embodiments, the disease or condition associated with the ACLY/Acetyl-CoA metabolic pathway is a cancer, high cholesterol, inflammation, atherosclerotic cardiovascular disease (ASCVD), nonalcoholic fatty liver disease (NAFLD), or cancer-associated fibrosis. In some embodiments, the disease or condition associated with the ACLY/Acetyl-CoA metabolic pathway is high cholesterol. In some embodiments, the disease or condition associated with the ACLY/Acetyl-CoA metabolic pathway is inflammation. In some embodiments, the disease or condition associated with the ACLY/Acetyl-CoA metabolic pathway is ASCVD. In some embodiments, the disease or condition associated with the ACLY/Acetyl-CoA metabolic pathway is NAFLD. In some embodiments, the disease or condition associated with the ACLY/Acetyl-CoA metabolic pathway is cancer-associated fibrosis.

In some embodiments, the disease or condition associated with the ACLY/Acetyl-CoA metabolic pathway is a cancer. In some embodiments, the cancer is acute myeloid leukemia (AML), chronic myeloid leukemia (CML), chronic lymphocytic leukemia (CLL), acute lymphoblastic leukemia (ALL), lymphoma, breast cancer, pancreatic cancer, glioblastoma, or prostate cancer. In some embodiments, the cancer is AML. In some embodiments, the cancer is CIVIL. In some embodiments, the cancer is CLL. In some embodiments, the cancer is ALL. In some embodiments, the cancer is lymphoma. In some embodiments, the cancer breast cancer. In some embodiments, the cancer is pancreatic cancer. In some embodiments, the cancer is glioblastoma. In some embodiments, the cancer is prostate cancer.

In some embodiments, the assay is in silico computational modeling, a binding assay, an ACLY enzymatic activity assay, an ACLY phosphorylation assay, an ACLY-mediated acetyl-CoA assay, an ACLY/acetyl-CoA-mediated histone acetylation assay, or an ACYL/acetyl-CoA-mediated fatty acid and lipid synthesis assay. In some embodiments, the assay is in silico computational modeling. In some embodiments, the assay is a binding assay. In some embodiments the binding assay is a high throughput binding assay. In some embodiments, the assay is an ACLY enzymatic activity assay. In some embodiments, the assay is an ACLY phosphorylation assay. In some embodiments, the assay is an ACLY-mediated acetyl-CoA assay. In some embodiments, the assay is an ACLY/acetyl-CoA-mediated histone acetylation assay. In some embodiments, the assay is an ACYL/acetyl-CoA-mediated fatty acid and lipid synthesis assay.

In some embodiments, the compound inhibits the interaction of PIP₂ and/or PIP₃ to ACLY. In some embodiments, the compound inhibits the interaction of PIP₂ or PIP₃ to ACLY. In some embodiments, the compound inhibits the interaction of PIP₂ and PIP₃ to ACLY. In some embodiments, the compound inhibits the interaction of Lyn tyrosine kinase to ACLY. In some embodiments, the compound inhibits the interaction of both PIP₂ and Lyn tyrosine kinase to ACLY. In some embodiments, the compound inhibits the interaction of both PIP₃ and Lyn tyrosine kinase to ACLY.

In some embodiments, the compound inhibits the activity a complex of PIP₂/Lyn tyrosine kinase/ACLY. In some embodiments, the compound inhibits the activity a complex of PIP₃/Lyn tyrosine kinase/ACLY.

The present disclosure also provides methods of treating a disease or condition associated with the ACLY/Acetyl-CoA metabolic pathway in a cell in a subject in need thereof. In some embodiments, the methods comprise administering to the subject a Src protein tyrosine kinase inhibitor (such as a Lyn tyrosine kinase inhibitor), an ACLY inhibitor, and a PI3K inhibitor to the subject. Any of the Lyn tyrosine kinase inhibitors, ACLY inhibitors, and PI3K inhibitors described herein, or any combinations thereof, in any of the amounts described herein can be used. The disease or condition associated with the ACLY/Acetyl-CoA metabolic pathway can be any of those described herein.

In some embodiments, the Lyn tyrosine kinase inhibitor, the ACLY inhibitor, and the PI3K inhibitor are administered to the subject together in a single pharmaceutical composition. In some embodiments, the Lyn tyrosine kinase inhibitor, the ACLY inhibitor, and the PI3K inhibitor are administered to the subject in separate compositions either simultaneously (i.e., within minutes of each other) or sequentially in any order.

The present disclosure also provides combinations of a Lyn tyrosine kinase inhibitor, an ACLY inhibitor, and a PI3K inhibitor for use in the manufacture of a medicament for treating a disease or condition associated with the ACLY/Acetyl-CoA metabolic pathway in a cell. Any of the Lyn tyrosine kinase inhibitors, ACLY inhibitors, and PI3K inhibitors described herein, or any combinations thereof, in any of the amounts described herein can be used. The disease or condition associated with the ACLY/Acetyl-CoA metabolic pathway can be any of those described herein.

The present disclosure also provides uses of a pharmaceutical composition comprising a Lyn tyrosine kinase inhibitor, an ACLY inhibitor, and a PI3K inhibitor for treating a disease or condition associated with the ACLY/Acetyl-CoA metabolic pathway in a cell. Any of the Lyn tyrosine kinase inhibitors, ACLY inhibitors, and PI3K inhibitors described herein, or any combinations thereof, in any of the amounts described herein can be used. The disease or condition associated with the ACLY/Acetyl-CoA metabolic pathway can be any of those described herein.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

The compounds described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: General Methodology

### Cells

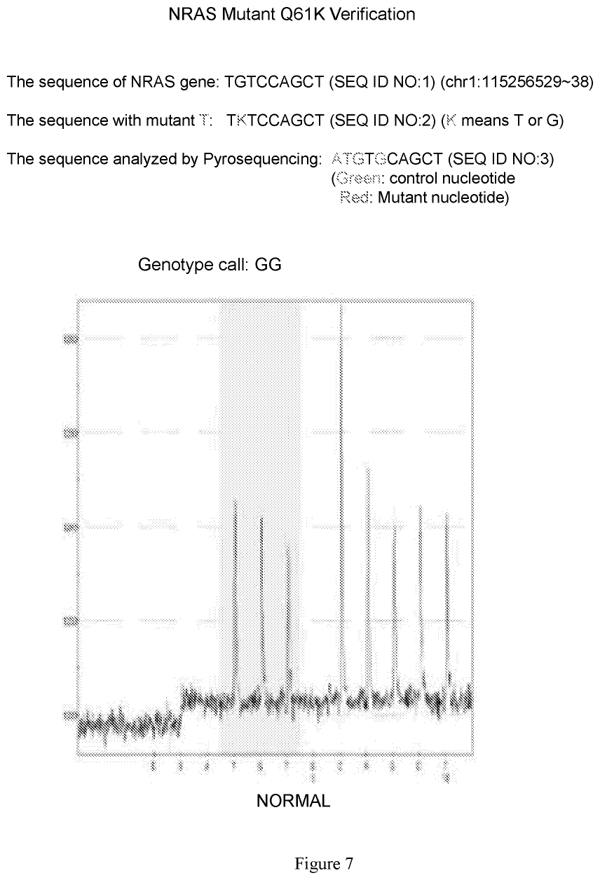

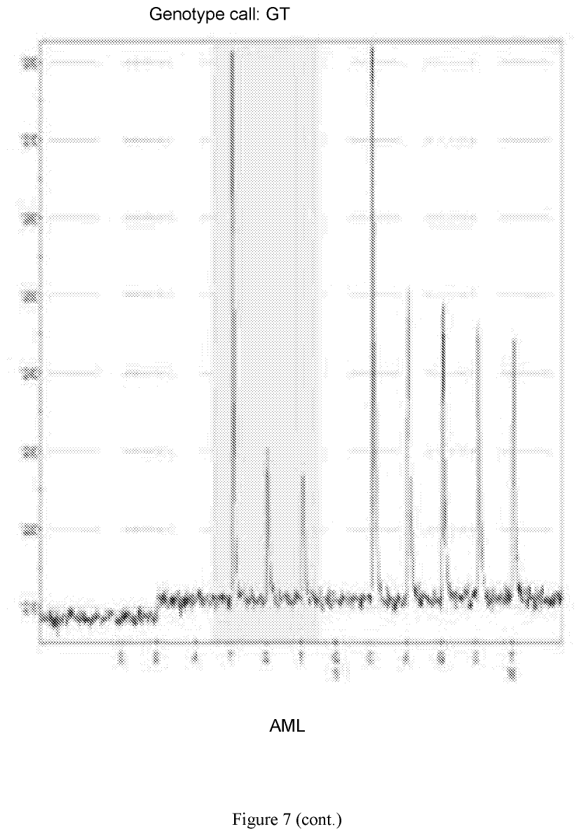

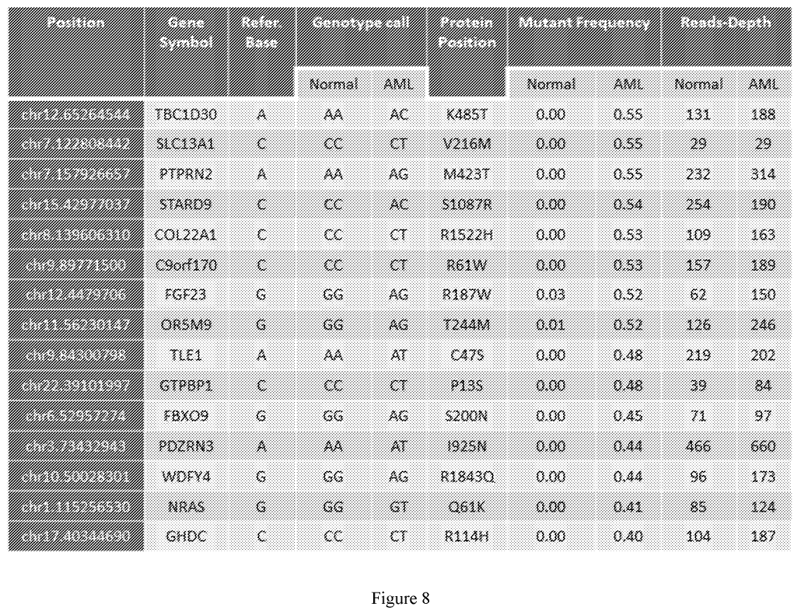

To investigate mechanistically connected signaling phenomena, in the experiments two different types of primary cells and two relevant cell lines: acute myeloid leukemia (AML) patient derived marrow cells, normal donor-derived CD34+ stem/progenitor marrow cells, HL-60 AML cell line and HEK293T human embryonic cell line transfected with ACLY alone or ACLY and Src were used. The AML patient's cells used herein contained the mutated NRAS, in addition to several other potentially PI3K pathway-activating mutated proteins and chromosomal aberrations (description of chromosomal aberrations and genetic pathogenic mutations is included in FIG. 7 and FIG. 8). Similarly, HL-60 AML cell line used herein has an oncogenic NRAS and high level of total Lyn tyrosine kinase activity and Lyn-associated PI3K activity, as compared to normal cells.

### AML Patient-Derived Marrow Cells

Patient. The patient was diagnosed in 2011 with the aggressive form of acute myeloid leukemia. A hypercellular marrow was extensively involved (75%) by cells with morphologic and immunophenotypic features of AML. Myeloid elements were markedly increased and left shifted, with only limited maturation. Immunostainings were performed on the bone marrow core with adequate controls. These showed that leukemia blasts were CD34+ MPO+ lysozyme+ cKIT+ and TdT−. No lymphoid aggregates were identified. There was no overt evidence of marrow involvement by lymphoma. Flow cytometry performed on the bone marrow aspirate demonstrated a discrete expansion of CD4+ CD13+ CD34+ CD33+ CD56+ CD117(dim var)+ HLA-DR+ blasts (41% of total events). A small subset of these cells (2-6% of total events) appeared to express B lineage markers CD19, CD22, and CD79a. There was also an expansion (25% of total cellularity) of atypical immature monocytes with the following dominant immunophenotype: CD4+ CD11b(var)+ CD13+ CD14(var)+ CD15+ CD34(dim)+ CD33+ CD56+ CD64+ HLA-DR+ MPO+. No population of light chain restricted B cells was identified. Cytogenetic studies showed the following abnormal male karyotype in 14 of 15 cells studied: 45,XY,t(3;3)(q21;q26),der(17)t(17;21)(p11.2;q11.2)(14)/46,XY. The patient was non-responsive to standard chemotherapeutic agents. As the last-ditch effort late in the disease, therapy with multi-kinase inhibitor Sunitinib was also initiated. However, the patient rapidly passed away with fulminant disease.

Genetic alterations. A mutational pattern of AML biopsy was analyzed with patient's normal cells serving as a control (standard methods of whole exome DNA sequence analysis WES and some of the selected mutations were additionally validated by pyrosequencing). The oncogenic nature of all mutations was evaluated by the algorithm predicting the functional effects of protein mutations, FATHMM-MKL, and through COSMIC and other databases. Several mutated proteins were identified that could potentially alter the PI3K pathway, including alterations of PIP₂ and PIP₃, in these AML cells, as compared to normal cells (as shown in FIG. 8 the mutant frequency is ≥0.4 in AML cells and ˜0.0 in normal cells). In addition to pathogenic DNA point mutations, chromosomal aberrations were identified that also could potentially contribute to alteration of PI3K pathway in these AML cells, as following:

NRAS: Q61K, this missense mutation has been reported in a variety of human solid and hematologic malignancies, including AML, and is described in a COSMIC database in detail. Mutations which change amino acid 61 activate the potential of RAS as they lock RAS proteins into a constitutively activated state in which they signal to downstream effectors, frequently PI3K. Consequently, the missense NRAS mutation position Q61K is pathogenic according to FATHMM score 0.993 (prediction scores are given in the range from 0 to 1 with scores >0.5 are predicted to be pathogenic). The presence of this mutation was confirmed by pyrosequencing (in addition to WES). The mutant Q61K verification, as compared to normal cells, is shown in FIG. 7.

FBXO9: S200N: F-box protein 9 is involved in pathway protein ubiquitination. The mutations of FBXO9 at various positions have been reported in non-hematological and hematological malignancies, including acute leukemia, according to COSMIC. The FBXO9 S200N mutation has not been yet reported in COSMIC. The S200N mutation is predicted to be pathogenic, FATHMM score is 0.940. It has been shown that overexpression of FBXO9 results in constitutive activation of the PI3K/mTORC2 pathway to promote survival in hematologic malignancies. Thus, the activating pathogenic mutations of FBXO9 can increase the PI3K activity.

TLE1-C47S: It is a transducin-like enhancer protein 1. Transcriptional corepressor that binds to a number of transcription factors, negative regulator of anoikis, negative regulator of I-kappaB kinase/NFkappaB signaling, negative regulator of Wnt signaling pathway. The mutations of TLE1 at various positions have been reported in solid tumors and hematologic malignancies, including acute leukemia, according to COSMIC. The TLE1 C47S mutation has not been yet reported in COSMIC. The C47S mutation is predicted to be pathogenic according to FATHMM (score 0.900). TLE1-regulated survival is directly mediated by PI3K and thus TLE1 mutations could affect the PI3K activity in AML cells.

TBC1D30-K485T: TBC1D30 is the TBC1 domain family member 30. It is a GTP-ase activating protein with broad specificity, mostly regulates Rab, but also may increase the activity of Rho, Ras, Rap, Cdc42 and Ran. Mutations of TBC1D30 at various positions have been reported in cancers, including acute leukemia, according to a COSMIC database. However, the particular TBC1D30 K485T missense mutation has not been yet reported in COSMIC. The K485T mutation is pathogenic, according to FATHMM (score 0.978). It was reported that downregulation of TBC1 domain family members inhibited breast carcinoma growth via PI3K pathways. The role of TBC1D30 in PI3K pathway in AML still needs to be determined.

PTPRN2: M423T mutation has been never reported in COSMIC. It has been suggested that PTPRN2 has phosphatidylinositol phosphatase activity rather that tyrosine phosphatase activity, but its precise function in signal transduction is still unclear. It was suggested that aberrant expression of PTPRN2 in cancer cells confers resistance to apoptosis. PTPRN2 is upregulated in glioma and highly metastatic breast cancer cells, and promote metastatic breast cancer migration through PIP₂-dependent mechanism. It was determined that PTPRN2 is also aberrantly expressed and upregulated in several malignant hematologic cell lines and AML primary cells, as compared to normal cells, but its precise role in AML phosphatidylinositol signal transduction still needs to be determined (data not shown).

Chromosomal aberrations and their potential effect on p53 and PI3K. It has been previously shown that the tumor suppressor p53 is located on the chromosome 17 and can inhibit the PI3K pathways through its effects on PTEN and AKT. P53 mutations occur in more than 50% cases in solid tumors, but only in less than 10% of AML cases. The particular chromosomal aberrations in our AML cells (45,XY,t(3;3)(q21;q26),der(17)t(17;21)(p11.2;q11.2)(14)/46,XY(1)), particularly that of chromosome 17, could potentially contribute to alteration of PI3K pathways due to perturbation or loss of p53 function, as described earlier in other cellular systems, and according to COSMIC.

HL-60 Cell Line (Treatment with Inhibitors of MK/LYN)

The limited viability of AML tissue in an in vitro culture, and poor propensity for transfection did not permit for methodical use of AML primary cells in some experiments in this project. Therefore, to evaluate the link between PI3K/Lyn and ACLY-mediated pathways in AML, HL-60 AML cell line were also used, in addition to primary patient-derived AML cells. HL-60 cell line was from American Type Culture Collection (ATCC). It was decided to use this particular HL-60 cell line for consistency herein since HL-60 cells express the active NRAS oncoprotein, similarly like the patient-derived AML cells (FIG. 7 and FIG. 8). HL-60 AML cell line was grown in RPMI 1640 medium supplemented with 10% fetal bovine serum, 2 mM 1-glutamine and 1% penicillin-streptomycin and maintained at 37° C. and 5% CO₂. Cells were treated with the Lyn inhibitor (100 nM-1000 nM Bafetinib) or PI3K inhibitors (500 nM-2.5 μM LY294002 or 100 nM-2.0 μM BKM120) or vehicle (0.1% DMSO) in the presence of 10% FBS in RPMI media for 16 hours for Acetyl CoA measurement, phospholipid and histone acetylation analysis.

### HEK293T Cells (ACLY-SRC/LYN Phosphorylation Experiments)

To evaluate potential interaction of ACLY with Lyn, and Src family kinases in general, the human embryonic HEK 293T cells were used because they are widely used due to their reliable growth and propensity for transfection. HEK 293T cells were purchased from ATCC, anti-HA antibody and HA conjugated agarose beads were obtained from ThermoFisher, PA. Pan Tyrosine antibody (pY100) and p-Src Y416 were from Cell Signaling Technology, MA. HA tagged ACLY and SRC kinase constructs and DNAfectin a transfection reagent was acquired from Applied Biological Materials, Canada. 293T cells were plated on 10 cm dish with DMEM and 10% FBS and next day at around 80% confluence transfected with the ACLY and SRC kinase constructs. After overnight incubation in transfection reagent, the media was replaced with fresh DMEM and cultured for additional 48 hours before harvest. The cells were harvested and proceeded for the immunoprecipitation of HA (ACLY). Samples were subjected to immunoprecipitation using HA-conjugated agarose and both input (5%) lysates and agarose beads were analyzed by immunoblot using p-ACLY (pan Tyrosine Y100), HA and p-Src Y416 (p-Lyn Y396) antibodies.

To probe the potential interaction between ACLY and Lyn, in vitro tyrosine kinase assay was performed on purified ACLY protein and Lyn immunoprecipitates that was obtained from HL-60 AML cells. For the source of LYN kinase, immunoprecipitation of LYN was performed on HL-60 cell (treated with DMSO or Bafetinib—500 nM for 16 hours) protein lysates using anti-LYN antibody for overnight. The source of non phosphorylated HA tagged ACLY was expressed in HEK293T cells as described earlier and immunoprecipitated with anti-HA agarose conjugated beads and eluted with HA peptide. The purified ACLY protein and LYN IP complex were incubated in the presence of kinase buffer (50 mM Tris.HCl, pH 7.5, 10 mM MgCl₂, 1 mM sodium fluoride, 1 mM sodium orthovanadate, 1 mM DTT and 1.0 mM ATP) for 30 minutes at 30° C. The kinase reaction was terminated by heating the samples at 95° C. for 5 minutes and separated on a 10% gel by SDS-PAGE and followed by western blotting and probed with anti-pan Tyrosine, anti-ACLY, anti-LYN and anti-pSRC Y416 (pLYN Y396) antibodies overnight.

### Regents, Antibodies and ACLY, PI3K, LYN Inhibitors

Anti-ATP Citrate lyase (ACLY) antibody and anti-GAPDH was purchased from Protein tech, Chicago (Catalog number: 15421-1-AP), anti-PIP₂ and anti-PIP₃ antibodies was from ThermoFisher (catalog), Anti-Histone H3 and Anti-Histone H3K9 and H3K27 was from Cell Signaling Technology (Danvers, Mass., USA). Secondary HRP-conjugated antibodies and x-ray films Electrochemiluminescence (ECL) reagent and non-fat dry milk were from purchased from ThermoFisher, USA). ACLY inhibitor, BMS 303141 from Tocris chemicals, Bafetinib (INNO-406) a potent and selective Lyn inhibitor (Catalog number: S1369), PI3K inhibitors, LY294002 (Catalog No. S1105) and Buparlisib (BKM120) was purchased from Selleckchem (Houston, Tex., USA) were dissolved in dimethylsulphoxide (DMSO) to 10 mM stock concentration., PicoProbe Acetyl-CoA Fluorometric Assay Kit from Biovision. The 3-(4,5-dimethylthiazol-2-yl)-2,5-diphenyltetrazolium bromide (MTT) solution was purchased from Sigma-Aldrich (St. Louis, Mo.).

Cellular Assay with the Tri-Functional PI(4,5)P₂ and PI(3,4,5)P₃ Derivatives.

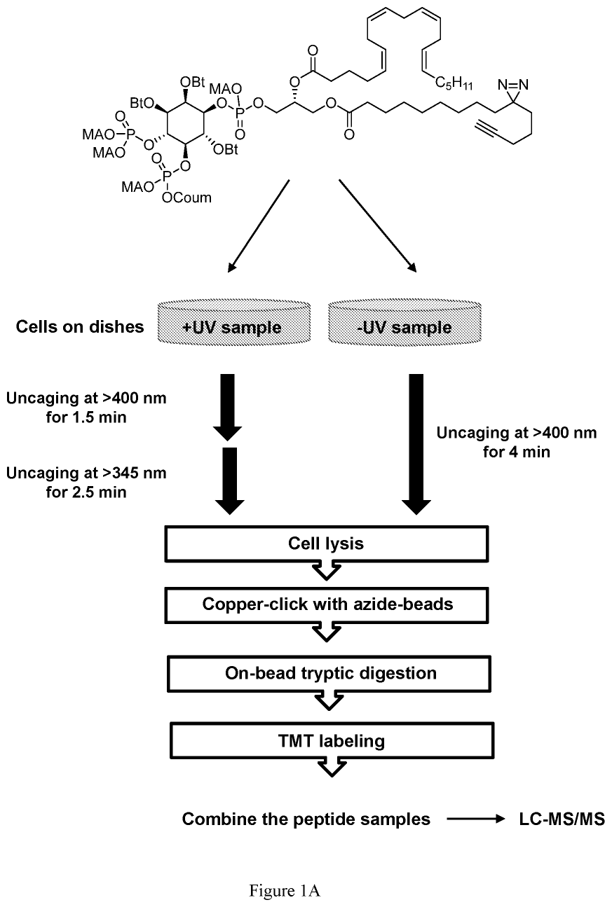

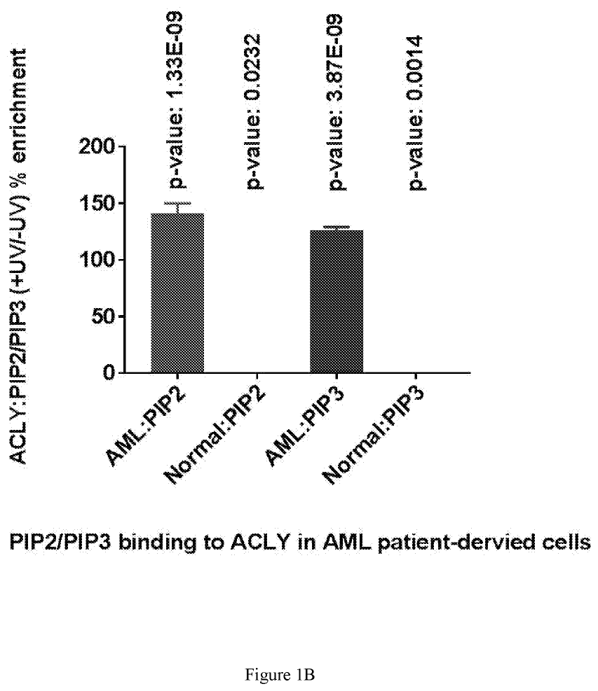

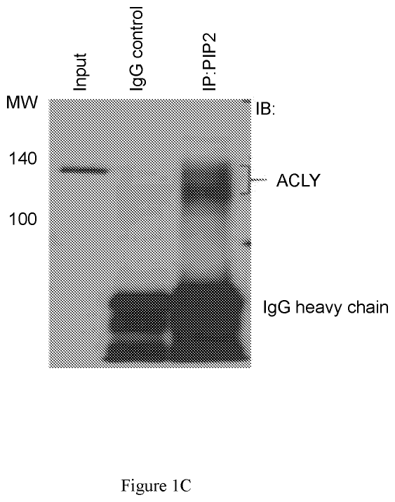

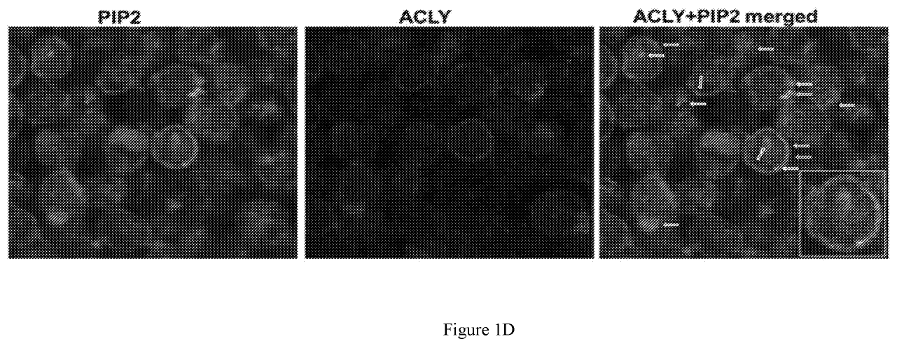

The Novel Tri-Functional PI(4,5)P₂ and PI(3,4,5)P₃ Membrane-Permeant Compounds were used. The chemical structure of the tri-functional compound, PI(4,5)P₂, is shown in FIG. 1A. The following modified following protocol was used. The Acute Myeloid Leukemia (AML) patient-derived marrow cells and normal donor-derived CD34+ stem/progenitor marrow cells in suspension were washed twice in serum-free RPMI 1640 by centrifugation, after which equal number of cells were resuspended in serum-free RPMI 1640, and were let adhere onto standard tissue culture-treated 60 mm dishes for 10 minutes at 37° C., 5% CO₂. The tri-functional PI(4,5)P₂ and PI(3,4,5)P₃ compounds from 10 mM DMSO-stocks were pre-mixed with 20% (w/v) Pluronic F-127 in DMSO in a 2:1 (v/v) ratio prior to addition to the cells.

Then the cells were fed with the compounds at 10 μM final concentration for 2 hours. At the end of the compound incubations, cells were washed once and illuminated under a 1000 W high-pressure mercury lamp (Newport, USA) equipped with two high-pass filters blocking wavelengths below 345 nm and below 400 nm. +UV samples were first illuminated at >400 nm for 1.5 minutes for coumarin uncaging to yield the metabolically active lipid, then were illuminated at >345 nm for 2.5 minutes for diazirine crosslinking to capture the protein binding partners. −UV samples were only illuminated at >400 nm for 4 minutes for coumarin uncaging. The −UV samples served as control samples to determine the background for the diazirine crosslinking.

Next, cells were directly lysed on dishes in lysis buffer (200 mM HEPES, 8 M Urea, 4% (v/v) CHAPS, 1 M NaCl, pH 8.0). After clearing the lysates, copper-catalyzed click reaction was performed overnight in presence of picolyl-azide agarose resin (Click Chemistry Tools). This step covalently captures lipid bound proteins onto the resin. The captured lipid-protein complexes on the beads were reduced by DTT in a boiling 2% (v/v) SDS buffer, and then alkylated by iodoacetamide in 2% SDS (V/V) buffer at room temperature. Following stringent washes at room temperature using 2% (v/v) SDS buffer, 8 M Urea buffer, 20% (v/v) acetonitrile (10× wash with each in the given order), the beads were subjected to tryptic digestion overnight. The digests were desalted on Sep-Pak tC18 columns (Waters). Desalted peptides were labeled with TMT reporter ions and combined, which was then subjected to liquid chromatography with tandem mass spectrometry analysis (LC-MS/MS).

Mass Spectrometric Identification of Protein in AML Marrow Blasts and Nonmalignant Marrow Cells Treated with the Tri-Functional PI(4,5)P₂ or PI(3,4,5)P₃ Derivatives.

Peptides were subjected to a reverse phase clean-up step (OASIS HLB 96-well μElution Plate, Watersv #186001828BA). Peptides were reconstituted in 10 μl 100 mMHepes/NaOH pH 8.5 and reacted with 80 μg of TMT10plex (Thermo Scientific, #90111) label reagent dissolved in 4 μl of acetonitrile for 1 hour at room temperature. Excess TMT reagent was quenched by the addition of 4 μl of an aqueous solution of 5% hydroxylamine (Sigma, 438227). Mixed peptides were subjected to a reverse phase clean-up step and analyzed by LC-MS/MS on a Q Exactive Plus (ThermoScentific). Briefly, peptides were separated using an UltiMate 3000 RSLC (Thermo Scientific) equipped with a trapping cartridge (Precolumn; C18 PepMap 100, 5 lm, 300 lm i.d.×5 mm, 100 A°) and an analytical column (Waters nanoEase HSS C18 T3, 75 lm×25 cm, 1.8 lm, 100 A°). Solvent A: aqueous 0.1% formic acid; Solvent B: 0.1% formic acid in acetonitrile (all solvents were of LC-MS grade). Peptides were loaded on the trapping cartridge using solvent A for 3 minutes with a flow of 30 μl/minute. Peptides were separated on the analytical column with a constant flow of 0.3 μl/minute applying a 1 hour gradient of 2-28% of solvent B in A, followed by an increase to 40% B. Peptides were directly analyzed in positive ion mode applying with a spray voltage of 2.3 kV and a capillary temperature of 320° C. using a Nanospray-Flex ion source and a Pico-Tip Emitter 360 lm OD×20 lm ID; 10 lm tip (New Objective). MS spectra with a mass range of 375-1.200 m/z were acquired in profile mode using a resolution of 70.000 (maximum fill time of 250 ms or a maximum of 3e6 ions (automatic gain control, AGC)). Fragmentation was triggered for the top 10 peaks with charge 2-4 on the MS scan (data-dependent acquisition) with a 30 second dynamic exclusion window (normalized collision energy was 32). Precursors were isolated with a 0.7 m/z window and MS/MS spectra were acquired in profile mode with a resolution of 35,000 (maximum fill time of 120 ms or an AGC target of 2e5 ions).

Acquired data were analyzed using IsobarQuantand Mascot V2.4 (Matrix Science) using a reverse UniProt FASTA Homo sapiens database (UP000005640) including common contaminants. The following modifications were taken into account: Carbamidomethyl (C, fixed), TMT10plex (K, fixed), Acetyl (N-term, variable), Oxidation (M, variable) and TMT10plex (N-term, variable). The mass error tolerance for full scan MS spectra was set to 10 ppm and for MS/MS spectra to 0.02 Da. A maximum of 2 missed cleavages were allowed. A minimum of 2 unique peptides with a peptide length of at least seven amino acids and a false discovery rate below 0.01 were required on the peptide and protein level.

Mass Spectrometric Data Analysis. The protein.txt output files of IsobarQuant were processed with the R programming language (ISBN 3-900051-07-0). As a quality filter, only proteins which were quantified with at least two unique peptides in both replicates were considered for the analysis. In total, 397 proteins passed these two criteria. The ‘signal sum’ columns (raw tmt reporter ion intensities) were first batch-cleaned using the ‘removeBatchEffect’ function of the limma package (PMID: 25605792) and then normalized using a variance stabilization normalization (vsn-PMID: 12169536). A separate normalization was performed for AML plusUV, AML minusUV and normal samples in order to keep the abundance differences between these conditions. Limma was employed again to test for differential expression between plusUV and minusUV of the various experimental conditions. Proteins were classified as ‘hit’ proteins with a false discovery rate smaller 5% and a fold-change of at least 100% and classified as ‘candidate’ proteins with a false discovery rate smaller 50% and a fold-change of at least 50%.

### Acly Binding to PI(4,5)P₂ in the PIP Specificity Plates Assay

Material: Human ACLY, His-tagged protein was obtained from Sino Biological. Membrane lipid strips and Cova PIP specificity plate were acquired from Echelon Biosciences. Active recombinant Src kinase and Lyn kinase, Anti-Biotin with HRP conjugated antibody, Super-Signal ELISA-Pico chemiluminescent substrate kit, were purchased from ThermoFisher. Malic dehydrogenase (MDH), potassium citrate, MgCl₂, DTT, CoA, ATP, and NADH were purchased from Sigma chemicals.

ACLY peptides: Based on PIP₂ binding motif analysis on ACLY using full length protein sequence, two PIP₂ binding domains were predicted. The ACLY peptide-1 in the ATP-grasp domain sequence (peptide-1: LVVKPDQLIKRRGKLG; SEQ ID NO:15) and the ACLY peptide-2, in the CoA-binding domain sequence (peptide-2: ALTRKLIKKADQKGV; SEQ ID NO:5). N-terminally biotinylated peptides were synthesized (Genscript, Inc). ACLY-PIP specificity binding assay was performed using the N-terminally biotinylated peptides at 1.0 μg/ml concentration in 1% goat serum in Tris buffer saline (TB S) and the Cova PIP Specificity plate H-6300 (Echelon Biosciences) according to manufactures instructions. In brief, ACLY peptide-1 and peptide-2 at final concentration of 1.0 μg/ml in 100 μl volume were incubated for overnight at 4° C. After three washes with TBST buffer, the wells were incubated with 100 μl of HRP-conjugated Biotin antibody, followed by three washes. The bound peptides were detected with Super-Signal ELISA-Pico chemiluminescent substrate kit from ThermoFisher and reading the absorbance at 450 nm for 3-30 minutes.

Acly Binding to PI(4,5)P₂ and PI(3,4,5)P₃ in the Membrane Lipid Strips Assay

For ACLY full length protein and lipid binding, hydrophobic membranes spotted with 100 pmol of fifteen different biologically important lipids found in cell membrane (Echelon Biosciences, P-6002) were purchased and binding assays were performed according to the manufacturer's instructions. Briefly, strips were blocked with 3% fatty acid-free BSA in PBS containing 0.05% Tween 20 (PBST) for 1 hour at room temperature and then incubated with 0.5 μg/ml of purified ACLY protein for overnight at 4° C. Next day, lipid strips were washed three times with PBST for 10 minute intervals and incubated in anti-ACLY (1:500) prepared in 3% BSA in PBST for overnight. Again, the strips were washed three times with PBST and protein binding was visualized using a HRP-conjugated anti-rabbit secondary antibody and ECL followed by developing a film. The ACLY bound to lipid strips were quantified using IMAGE software to analyze densitometry values for quantitation using PRISM Graphpad statistical analysis tool.

### Phosphoproteomics Analysis of Acly In Vitro Phosphorylated Samples—Identification of the Novel Lyn/Src-Dependent Phosphorylation Sites

To identify ACLY is a substrate of Src and Lyn (Src family kinase) for tyrosine phosphorylation, in vitro tyrosine kinase assay was performed on bacterially expressed and purified recombinant full length ACLY protein. In brief, 3.0 μg of ACLY and 100 ng of Src or Lyn kinase were incubated for 30 minutes at 30° C. in kinase buffer (25 mM Tris.HCl, pH 7.5, 150 mM NaCl, 1 mM DTT, 0.01% NP-40, 10 mM MgCl₂ and 0.2 mM ATP) in final volume of 50 μl. The reaction was terminated by adding 10 μl of 6×SDS sample loading buffer and heating the samples at 95° C. for 6 minutes.

To confirm the tyrosine phosphorylation of ACLY, SDS-PAGE and western blotting were performed on fraction of (5 μl) in vitro kinase phosphorylated samples and probed with pan-tyrosine (pY100) antibody. After that the remaining samples were separated on 10% novexNuPAGE (Invitrogen) and stained with colloidal blue for overnight. The bands were excised with clean and sterile blade and collected in Eppendorf tubes.

The gel band was destained with 100 mM Ammonium bicarbonate/acetonitrile (50:50). The band was reduced in 10 mM DTT/100 mM ammonium bicarbonate for over 60 minutes at 52° C. Then, the band was alkylated with 100 mM iodoacetamide in 100 mM ammonium bicarbonate at room temperature for 1 hour in the dark. The proteins in the gel band were digested by incubation with trypsin overnight. The supernatant was removed and kept in fresh tubes. Additional peptides were extracted from the gel by adding 50 μL of 50% acetonitrile and 1% TFA and shaking for 10 minutes. The supernatants were combined and dried. The dried samples were reconstituted by 0.1% formic acid for mass spectrometry analysis. Desalted peptides were analyzed on a Q-Exactive HF (Thermo Scientific) attached to an Ulimate 300 nano UPLC system (Thermo Scientific). Peptides were eluted with a 25 minute gradient from 2% to 32% ACN and to 98% ACN over 5 minutes in 0.1% formic acid. Data dependent acquisition mode with a dynamic exclusion of 45 second was enabled. One full MS scan was collected with scan range of 350 to 1200 m/z, resolution of 70 K, maximum injection time of 50 ms and AGC of 1e6. Then, a series of MS2 scans were acquired for the most abundant ions from the MS1 scan (top 15). Ions were filtered with charge 2-5. An isolation window of 1.40 m/z was used with quadruple isolation mode. Ions were fragmented using higher-energy collisional dissociation (HCD) with collision energy of 28%. Orbitrap detection was used with, resolution of 35 K, maximum injection time of 54 ms and AGC of 5e4.

Peptide Identification using Database Search: Proteome Discoverer 2.3 (Thermo Scientific) was used to process the raw spectra. Database search criteria were as follows: taxonomy Homo sapiens, carboxyamidomethylated (+57 Da) at cysteine residues for fixed modifications, oxidized at methionine (+16 Da) residues, phosphorylation (+79.9 Da) at serine, threonine, and tyrosine residues for variable modifications, two maximum allowed missed cleavage, 10 ppm MS tolerance, a 0.02-Da MS/MS tolerance. Only peptides resulting from trypsin digestion were considered. The target-decoy approach was used to filter the search results, in which the false discovery rate was less than 1% at the peptide and protein level.

### Western Blotting

HL-60 cell line was seeded at a concentration of 1×10⁶/ml in 10 ml media onto 40 ml culture-flask and cultured for 16 hours in the presence of indicated concentration of LYN or PI3K inhibitors or DMSO (0.1% dimethyl sulfoxide). Cells were harvested and lysed in 1 ml of ice-cold Pierce IP lysis buffer (Pierce Inc.) in the presence of Proteinase and phosphatase inhibitor cocktail (Thermofisher) and sonicated for 20 seconds. Lysates were cleared by centrifugation at 12000 rpm in cold conditions on bench top centrifuge, and the supernatant was used for protein determination by BCA assay kit (Pierce Inc). Equivalent amounts of protein lysate were mixed with sodium dodecylsulfate (2×SDS) and boiled for 8 minutes. Samples were resolved by 4-12% gradient sodium dodecylsulfate-polyacrylamide gel electrophoresis (NOVEX; Invitrogen) and blotted onto nitrocellulose filters. Membranes were blocked for 1 hour in 0.1% Tween-20 and 5% bovine serum albumin. Primary antibodies for H3K9ac, histone H3, Lyn, ACLY and GAPDH were added to 5% BSA at concentrations provided by the vendor's instructions and incubated with membranes overnight at 4° C. before removing by washing. Horseradish peroxidase linked-secondary antibody in 5% BSA was added for 1 hour before washing and signal detection using Super Signal West Pico Chemiluminescent Substrate (Thermo Scientific).

Immunoprecipitation of Phosphatidylinositol 4,5-Bisphosphate (PIP₂) in HL-60 cells and Western blotting against ACLY: The immunoprecipitation (IP) protocol was carried out using the magnetic Dynabeads conjugated with Protein A/G (Thermofisher). For IP, 2 mg of HL-60 cell lysate protein was incubated with 4 μg of anti-PIP₂ mouse primary antibody and IgG control at 4° C. overnight. Immunoprecipitated samples were washed four times with lysis buffer and eluted with 2× Laemmli sample buffer and incubated at 95° C. for 5 minutes. The samples were resolved on protein gel electrophoresis in 8% Bis-Tris gels in a Novex mini-gel system (Invitrogen). Separated proteins were transferred onto nitrocellulose membranes using BIORAD transfer apparatus. Western blotting with ACLY antibodies was subsequently carried out as describe above.

### Immunofluorescence

HL-60 cells were fixed with 4% paraformaldehyde (PFA) for 10 minutes. For PIP₂ and ACLY staining, cells were permeabilized with 0.01% Triton X-100 in PBS, followed by incubation with blocking solution, containing PBS supplemented with 5% bovine serum (for 30 minutes at room temperature) followed by overnight incubation with appropriate primary antibodies at 4° C. After three successive washes in PBS, the cells were incubated with secondary antibodies (Alexa Fluor 488 goat anti-mouse and Texas Red goat antirabbit) for 1-2 hours, washed three times in PBS, and cells were placed in tissue culture treated glass bottom 96 well plate for confocal microscopy. Fluorescence microscopy was performed on a Leica confocal microscope and the following filter sets were used: FITC (excitation: 490/520 nm, emission) and Texas Red (excitation: 590/620 nm, emission: 617/673).

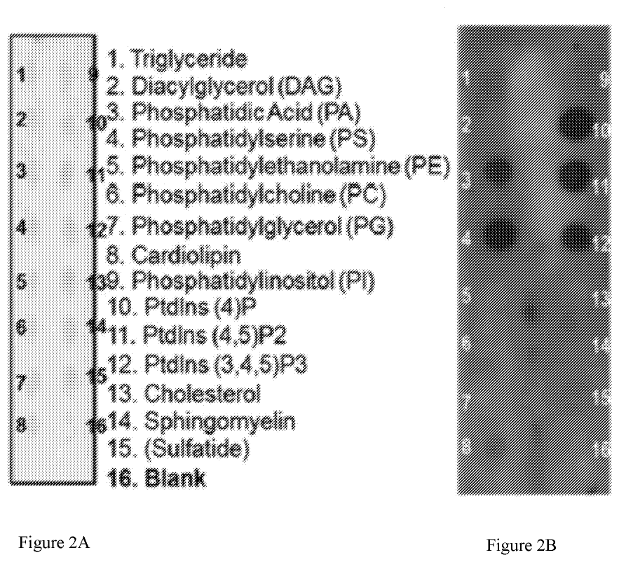

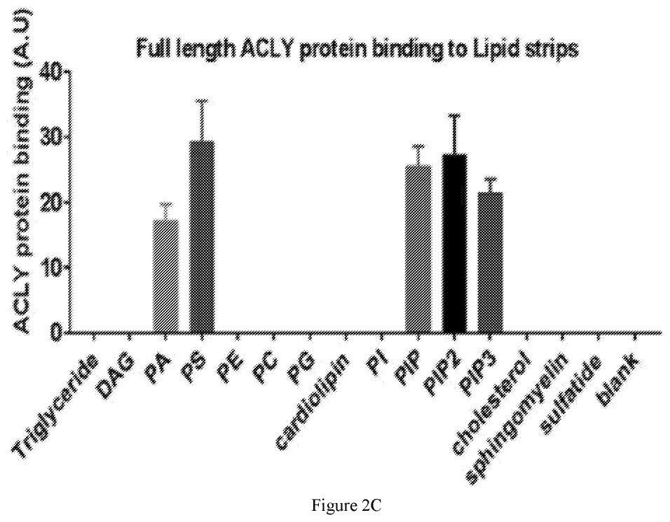

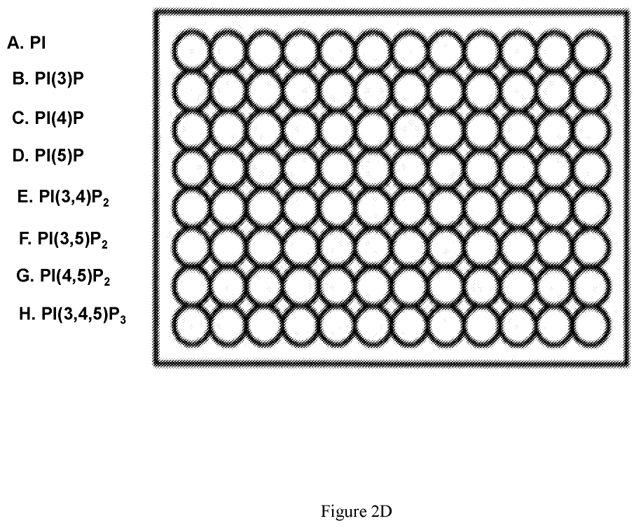

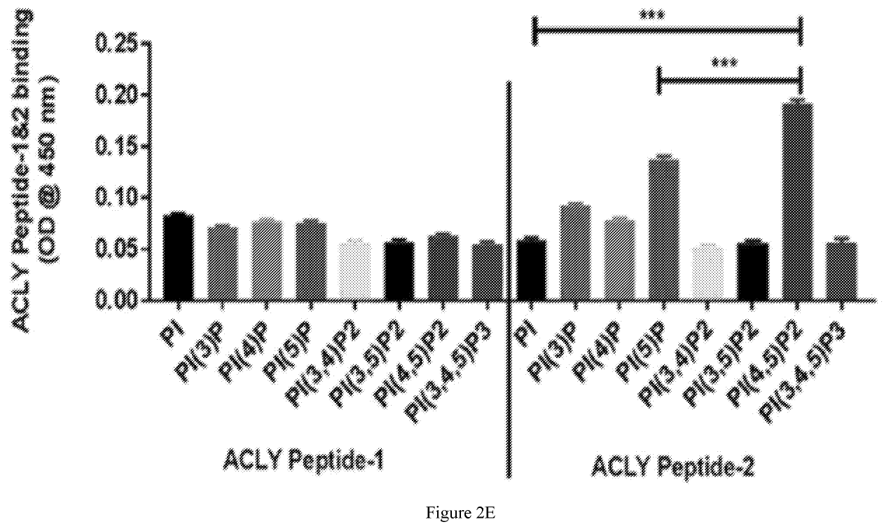

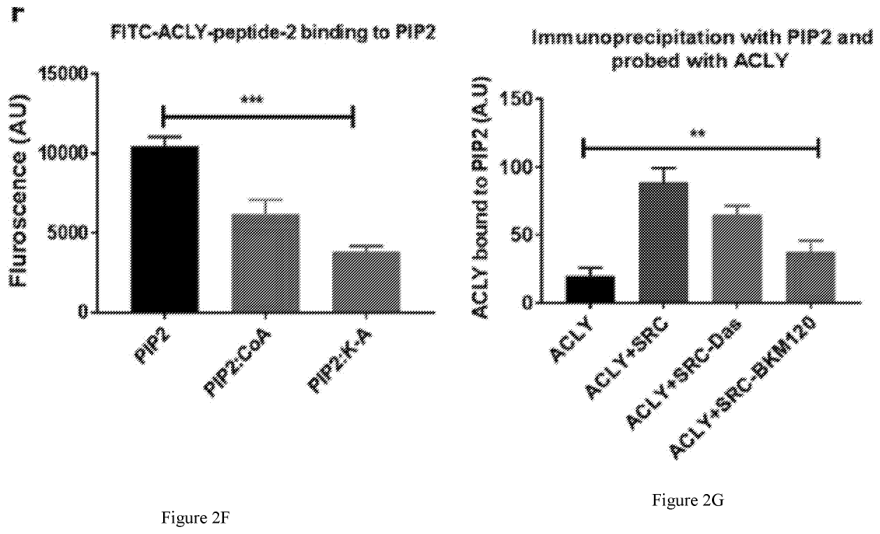

The endogenous basal PIP₃ level is several orders of magnitude lower than PIP₂ in living cells, its half-life is very short in stimulated cells, and is usually undetectable in unstimulated cells. For these reasons, colocalization of ACLY and endogenous PIP₃ in the cells was not detected by immunofluorescence (data not shown). However, a high ACLY enrichment by both exogenous PIP₂ and PIP₃ was obtained in the binding assays (FIG. 1B, FIG. 2), since the concentrations of the introduced exogenous PIP₂ and PIP₃ were identical during treatment of cells in this assay.

### Acetyl-CoA Measurement

HL-60 cells were cultured as above, treated with Bafetinib (500 nM), PI3K inhibitors (1 μM) for 16 hours. After treatment, cells were washed with medium and harvested for acetyl-CoA extraction from 5×10⁶ cells per condition in triplicate, acetyl-CoA levels were measured using Pico-Probe Acetyl-CoA Fluorometric Assay kit (BioVision, Milpitas, Calif.) following the manufacturer's instructions.

### Acly Enzyme Activity Measurement

ACLY enzyme activity was determined using the malate dehydrogenase (MDH)-coupled method as described earlier with little modification. Briefly, HL-60 cells 10×10⁶ cells were treated with DMSO or LYN kinase inhibitor, Bafetinib 1.0 μM and PI3Kinase inhibitor BKM120, 2.0 μM and AKT inhibitor, Capivasertinib (5 μM) for 16 hours and lysates were prepared. For ACLY activity, 50 μg of crude lysates for each condition in triplicate well (96 plate) were incubated in reaction buffer containing 10 mM potassium citrate, 10 mM MgCl₂, 1 mM DTT, 10 U malic dehydrogenase, 0.3 mM CoASH, 0.1 mM NADH in 50 mM Tris (pH 8.0) and the reaction was initiated by adding 0.2 mM ATP in a final volume of 100 μl, incubated at 37° C., and NADH oxidation was continuously monitored every 2 minutes for 60 minutes using a micro plate reader.

### MTT Assay

HL-60 cells were treated with various concentrations of Bafetinib (Lyn kinase), BKM120 (PI3K) and BMS 303141 (ACLY) inhibitors in the presence of 10% RPMI media for 72 hours. The growth-inhibitory effect was examined using a 3,4,-5-dimethyl-2H-tetrazolium bromide assay (MTT; Sigma-Aldrich) as per the instructions of the manufactured kit. The experiment was performed in triplicate.

### Lipidomic Analysis

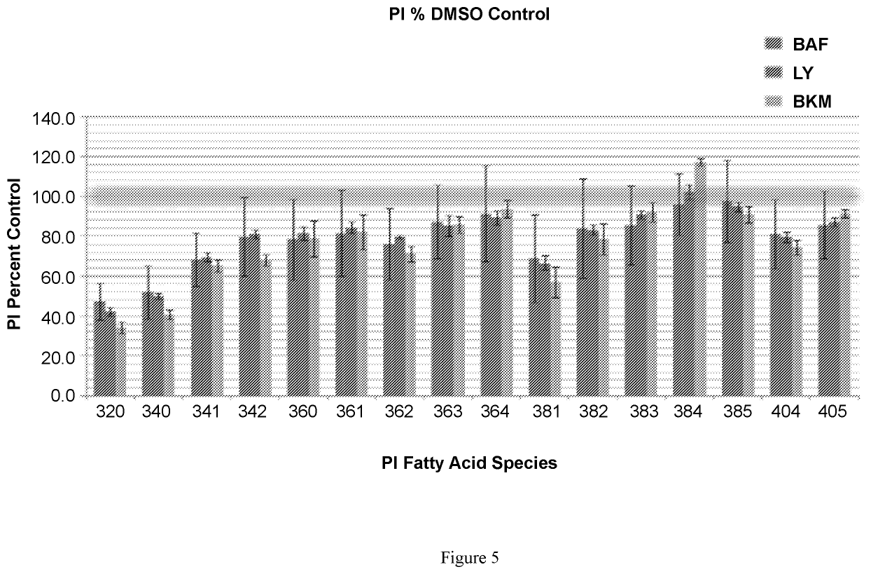

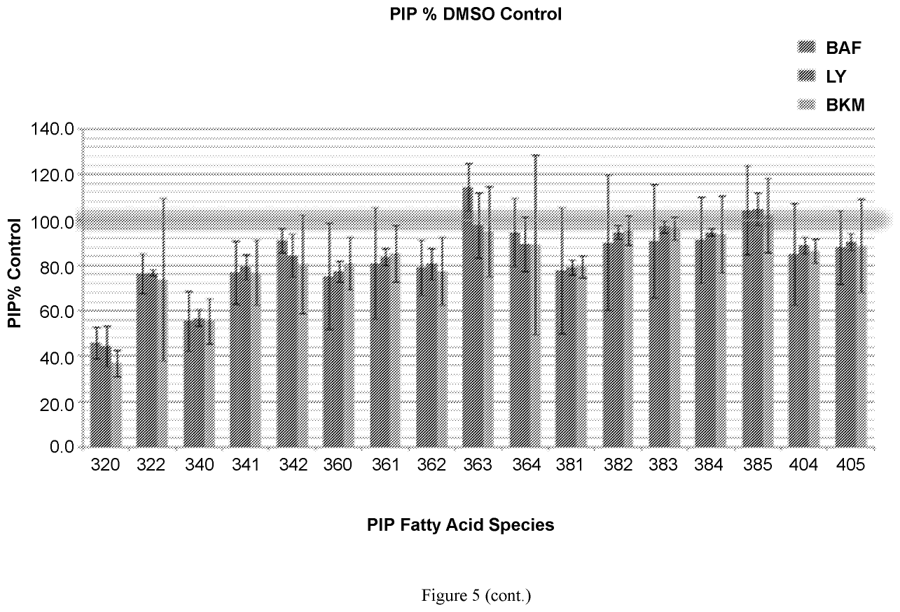

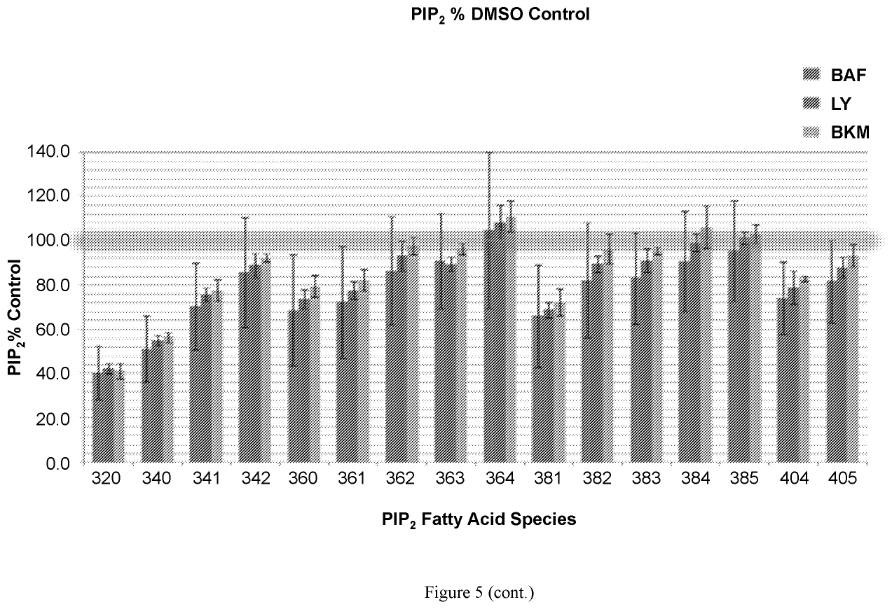

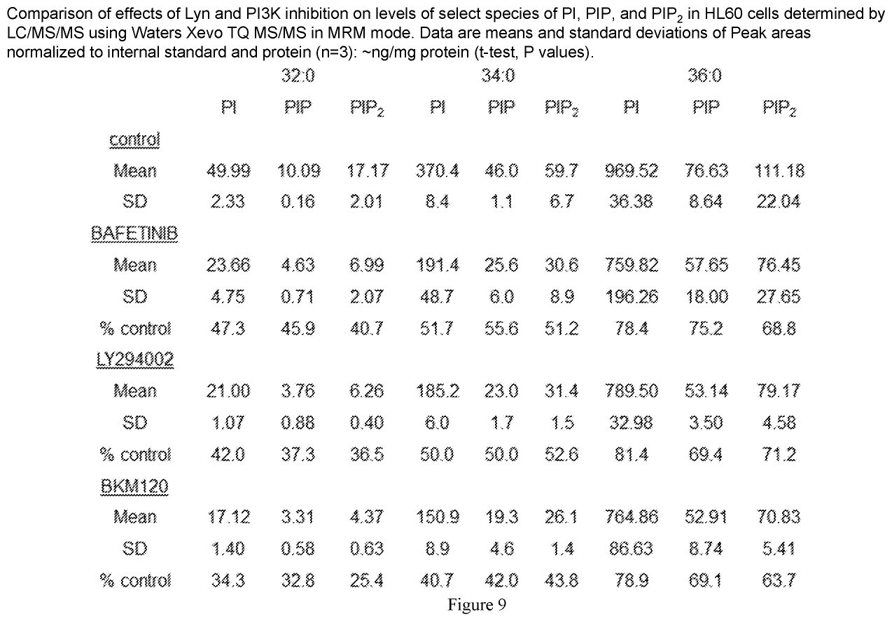

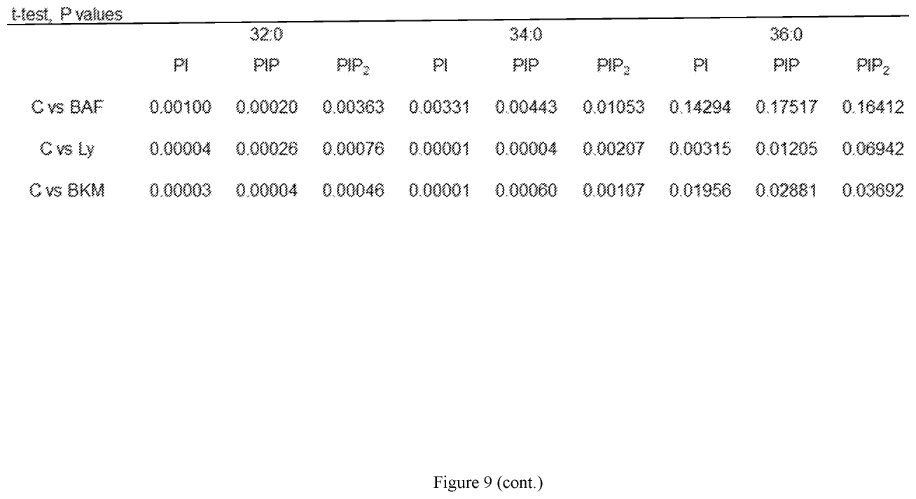

Phosphoinositides from HL60 cells treated with PI3K and Lyn inhibitors were compared with those from control cells (0.1% DMSO in culture medium). HL60 cells were treated with PI3K inhibitors LY294002, BKM120 or the Lyn inhibitor Bafetinib for 16 hours followed by TCA precipitation and freezing at −80° C. TCA precipitates were spiked with 20 ng of two internal standards (PIP 37:4; PIP₂ 37:4) and subjected to lipid extraction as described below and analyzed via LC/MS/MS. TMS-diazomethane derivatized phospholipids including phosphatidyl inositol (PI), phosphatidylinositol phosphate (PIP) and phosphatidylinositol bisphosphate (PIP₂) values were measured using UPLC/MS/MS MRM Mass Spectrometry. Several fatty acid species (notably, 34:1 and 36:1) of PI, PIP and PIP₂ were consistently lower with all 3 treatments (vs. DMSO), when data was normalized to internal standards and protein (FIG. 5 and FIG. 9). Phosphatidylinositol trisphosphate (PIP₃) was also measured but levels were too low in these cells to be analyzed. The endogenous PIP₃ is several orders of magnitude less abundant than PIP₂ in cells. It is well established that the basal PIP₃ level is often undetectable in cells due to its low quantity and short halflife.

Materials: Methanol, chloroform, dichloromethane, and acetonitrile (Fisher) were all of mass spectrometry grade. Sodium formate and HCl were from Sigma, and TMS-diazomethane (TMS-DM, 2.0 M in hexanes) from Sigma-Aldrich and Acros. The lipid analytical internal standards were ammonium salts of 1-heptadecanoyl-2-(5Z,8Z,11Z,14Z-eicosatetraenoyl)-sn-glycero-3-phospho-(1′-myo-inositol-3′,4′,5′-trisphosphate) (17:0, 20:4 PI(3,4,5)P₂), 1-heptadecanoyl-2-(5Z,8Z,11Z,14Z-eicosatetraenoyl)-sn-glycero-3-phospho-(1′-myoinositol-4′,5′-bisphosphate) (17:0, 20:4 PI(4,5)P₂); 1-heptadecanoyl-2-(5Z,8Z,11Z,14Z-eicosatetraenoyl)-sn-glycero-3-phospho-(1′-myo-inositol-4′-phosphate) (17:0, 20:4 PI(4)P) from Avanti Polar Lipids (LIPIDMAPS MS Standards), Alabaster, Al.

Lipid Extraction (acidic lipid extraction): Internal standard (PIP 37:4, 20 ng; PIP₂ 37:4, 20 ng, PIP₃ 37:4 2 ng) was added to the frozen pellet of TCA precipitates and then 670 μL of ice-cold chloroform/methanol/12.1 M HCl, 10/20/1, v/v/v was added. Samples were vortexed to fully resuspend and mix the pellet. An additional 650 μL of ice-cold chloroform was added to each sample and the tubes were vortexed after which 300 μL of 1 M HCl was added to each tube which were again vortexed for another 2 minutes, followed by centrifugation at 13,000 rpm for 2 minutes. The lower phases were collected into 2 mL fresh tubes and 1 ml of theoretical lower phase (chloroform/methanol/1.74 M HCl, 86/14/1, v/v/v) to the remaining upper phase and vortexed for 2 minutes and then centrifuged as before. The lower phase was collected and combined with the previously collected lower phase. Samples were spun again at 13,000 rpm for 2 minutes and the residual upper phase was removed. Samples were then evaporated to dryness under N₂ in a Biotage evaporator prior to methylation.

UPLC/MS: Dried, methylated cell extracts were suspended in 100 μl 100% methanol (LC-MS Optima grade, Fisher) prior to chromatographic separation and MS/MS. A Waters Acquity FTN autosampler set at 4° C. injected 5 μl of sample extract into the UPLC/MS. For chromatography over a C8 column, the mobile phase consisted of a 18 minute runtime at a flow rate of 0.3 ml/minute by a Waters Acquity UPLC (Waters Acquity UPLC Protein BEH C8, 1.9 μm 2.1×50). The gradient was initiated with 10 mM formic acid in water/10 mM formic acid in acetonitrile/methanol/isopropanol (35/10/5, v/v/v), (33:67 v/v), held for 1 minute, then increased to 15:85, v/v in 9 minutes following injection, held at 85% for 1 minute and then raised to 100% over 1 minute and held at 100% for 2 minutes and then reequilibrated to starting conditions for 3 minutes. The effluent was monitored by a Waters XEVO TQ-S MS/MS in multiple reaction monitoring (MRM) in positive ion mode. Sodium formate (50 μM in water/acetonitrile, 1/1, v/v) was infused into the post-column eluate using the Intellistart Fluidics of the Waters XEVO TQ-S MS/MS to promote formation of positively charged sodiated adducts.

### Example 2: ACLY Interacts with PIP₂/PIP₃ in Patient-Derived AML Cells

Because AML patient-derived blasts, in contrast to non-malignant myeloid cells, express multiple mutated proteins that can alter PI3K signaling (FIG. 8), whether the substrate and product of PI3K, PIP₂ and PIP₃, respectively, could bind to ACLY in these cells was examined. Investigations of PIP₂/PIP₃ actions are often hampered by a lack of tools that can be used in living cells. However, it has recently been demonstrated that the novel tri-functional lipid probes, including the phosphatidylinositol probes well represent the endogenous lipid and phosphatidylinositol pool in living cells. Thus, the association of PIP₂/PIP₃ with ACLY was probed by incubating AML and control cells with the tri-functional derivatives of PIP₂ and PIP₃ (FIG. 1A), and applying the properly normalized ACLY enrichment procedures and mass spectrometry analysis (FIG. 1B). ACLY was enriched by PIP₂ and PIP₃ more than 100% in AML patient blasts, while no enrichment was observed in illuminated non-malignant myeloid cells (FIG. 1B). These data show the direct association of PIP₂/PIP₃ with ACLY in living primary AML blasts. The association of PIP₂ with ACLY was confirmed in the HL-60 AML cell line by looking for ACLY in PIP₂ immunoprecipitates by Western blotting (FIG. 1C) and colocalization of ACLY with PIP₂ by immunofluorescence (FIG. 1D). PIP₃ was also measured, but the basal endogenous PIP₃ levels were too low in these cells to be analyzed by immunofluorescence or blotting. It is well established that the abundance of PIP₃ in living cells is several orders of magnitude lower than PIP₂. Therefore, the association of PI(3,4,5)P₃ was probed with ACLY by binding the ACLY full length protein to membrane lipid strips (the membranes were spotted with 100 pmol of fifteen biologically important lipids) (FIG. 2A). ACLY bound selectively to PIP, PIP₂ and PIP₃ in the membrane lipid strips binding assay (FIG. 2B and FIG. 2C). In contrast, no binding of ACLY to phosphatidylinositol (PI) and several other lipids was detected, under the same conditions (FIG. 2B and FIG. 2C). These data indicate that phosphorylated forms of phosphatidylinositol (PIP, PIP₂ and PIP₃), which are known to play important roles in cell signaling, can selectively interact with ACLY, in contrast to phosphatidylinositol (PI), which is their precursor and thus structurally very similar. It is consistent with the data obtained with the trifunctional PIP₂ and PIP₃ derivatives in living cancer cells (FIG. 1B).

### Example 3: Identification of the PIP₂ Binding Region of ACLY

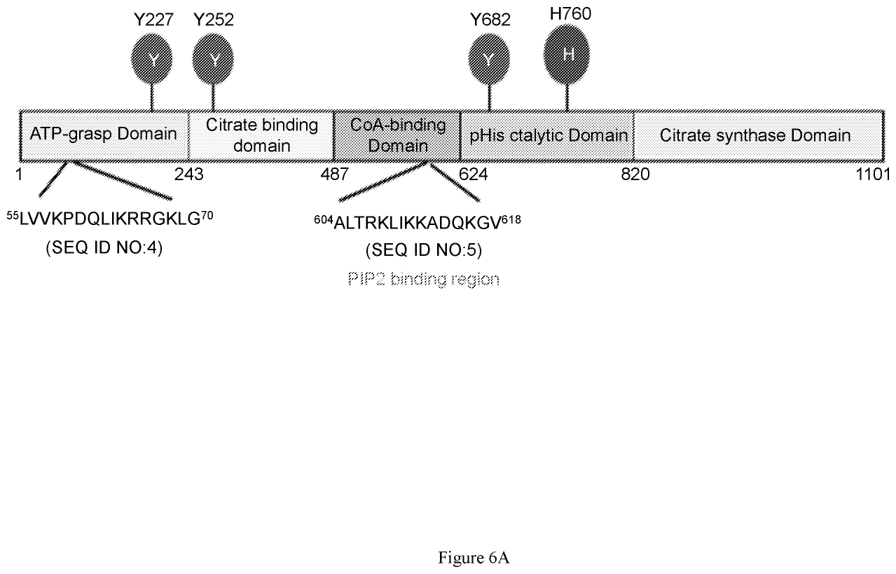

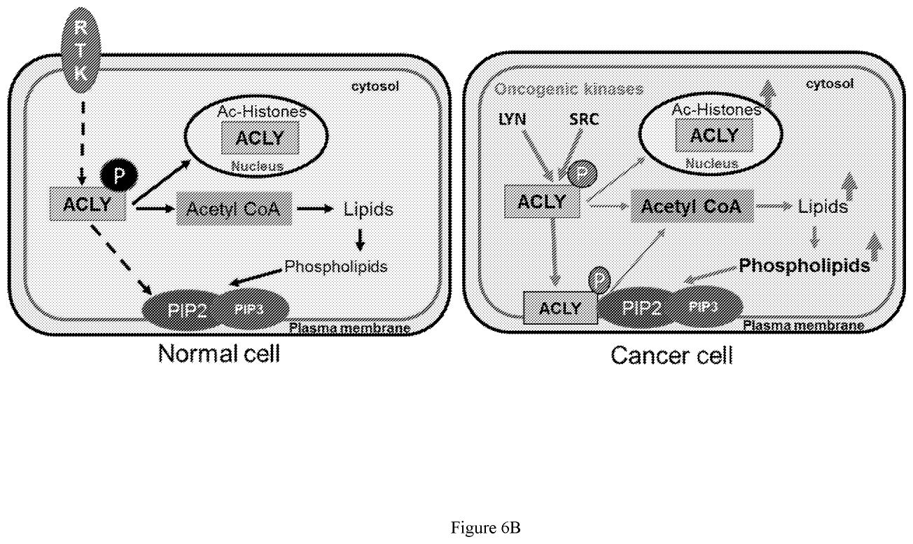

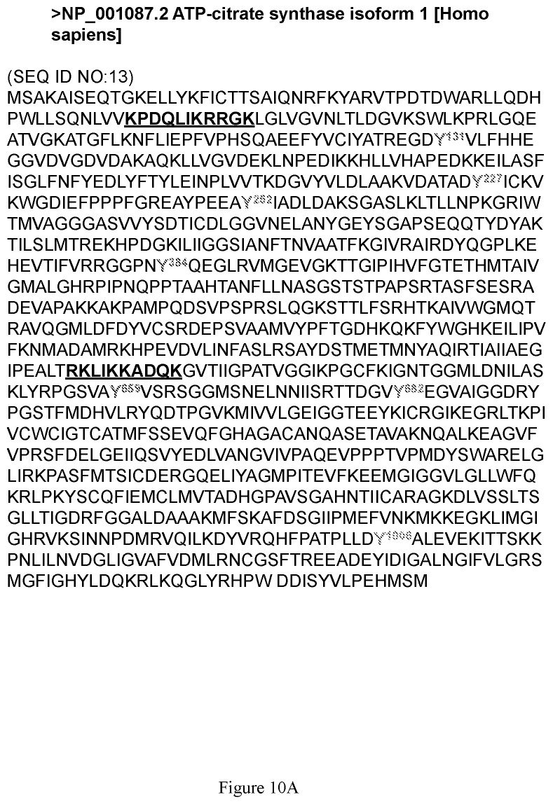

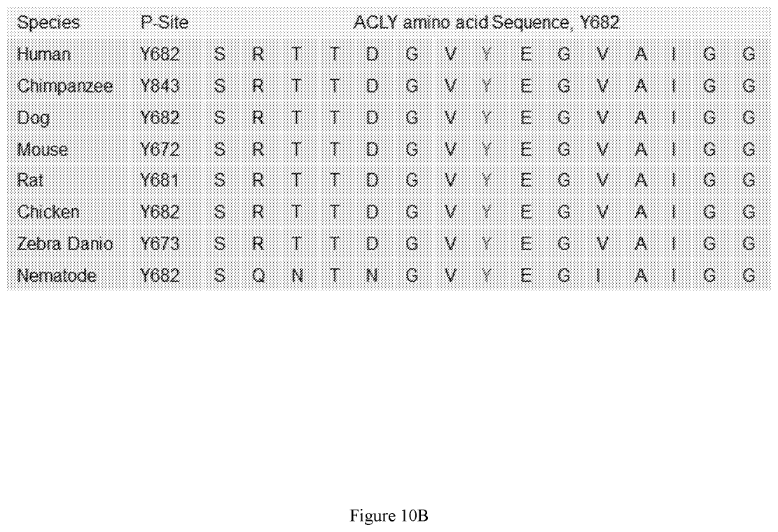

Based on the PIP₂ binding motif analysis and using the full length ACLY protein sequence, two potential PIP₂ binding regions were predicted: the ATP-binding domain and the CoA-binding domain of ACLY. Therefore, two different ACLY peptides were synthesized containing either the ATP-binding domain or the CoA-binding domain sequences (FIG. 6A and FIG. 10). The binding of these ACLY peptides to phospholipids on the Cova PIP

Specificity Plates (FIG. 2D and FIG. 2E) and the ACLY mutant experiment (FIG. 2F) indicated that PI(4,5)P₂ selectively bound to the CoA-binding domain (peptide-2), but not to the ATP-binding domain (peptide-1) of ACLY. The differences detected by this binding assay between PI(4,5)P₂ and seven other control phospholipids were highly statistically significant. The ACLY peptide binding results on the Cova PIP Specificity Plates were consistent with the data obtained with five other assays: 1) the trifunctional PIP₂/PIP₃ derivatives binding assay in living cancer cells (FIG. 1A and FIG. 1B), 2) protein co-immunoprecipitation by Western blotting (FIG. 1C), 3) protein co-localization by immunofluorescence (FIG. 1D), 4) membrane lipid strips binding assay (FIGS. 2A-2C), and 5) the phospho-ACLY binding to PIP₂ in transfected cells (FIG. 2G). Taken together, the mechanistically distinct experimental approaches and multiple data indicate consistently that ACLY directly binds to PIP₂ and PIP₃ and the specific association with PIP₂ is mediated through the ACLY CoA-binding domain (FIG. 2E and FIG. 2F).

### Example 4: ACLY is Phosphorylated on Tyrosine Residues by Lyn in AML

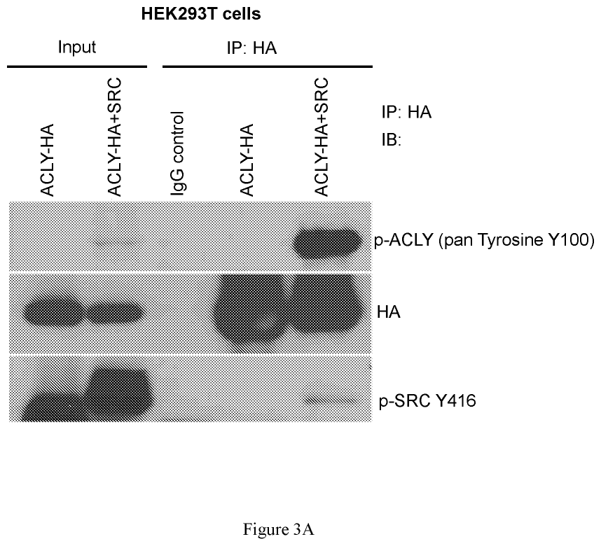

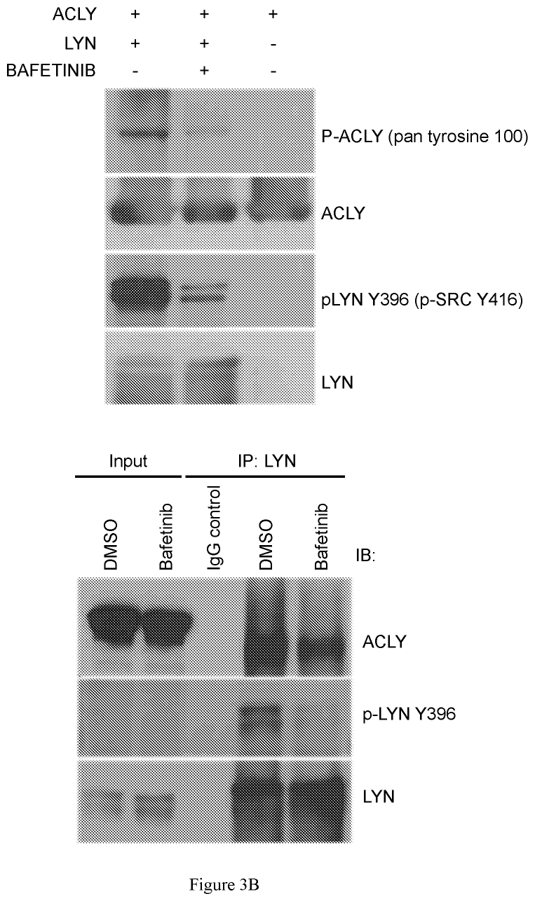

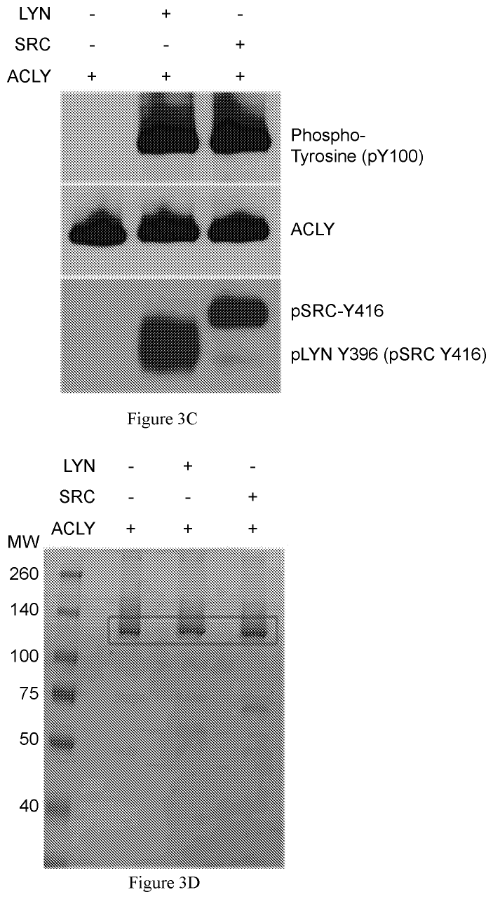

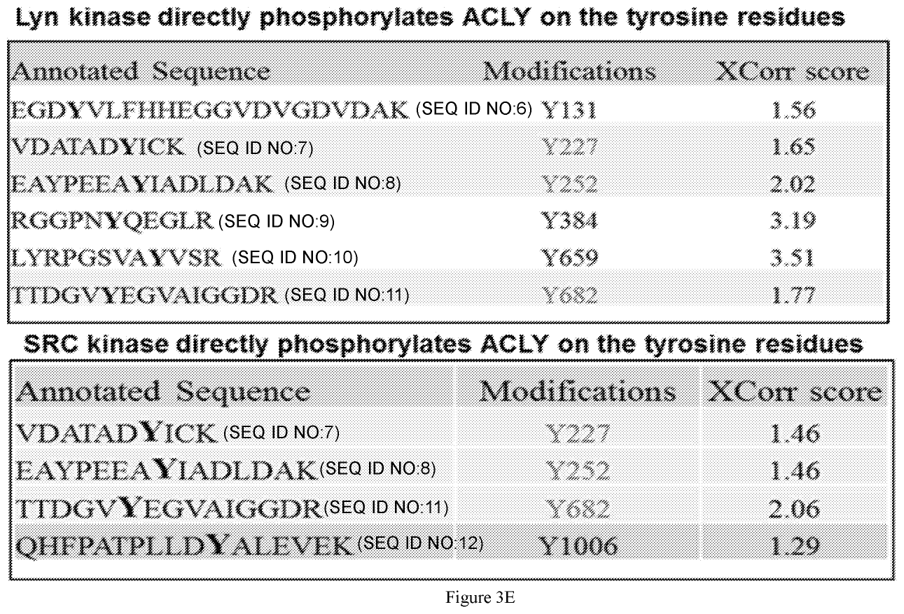

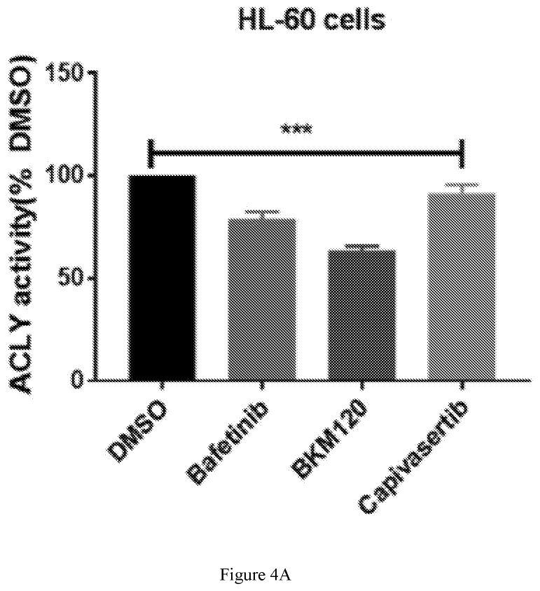

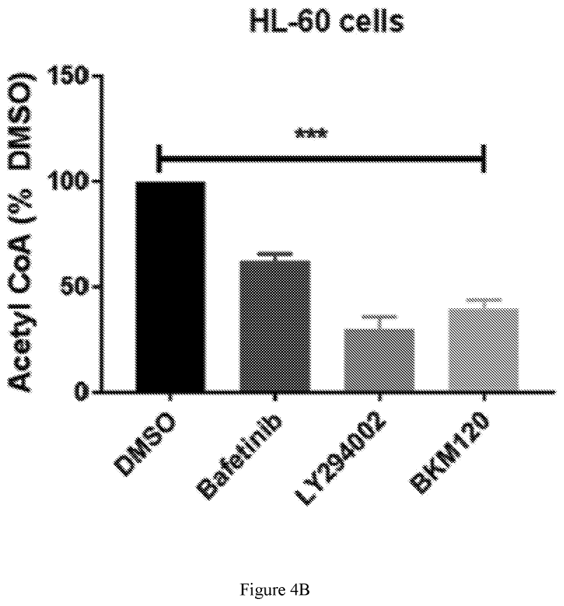

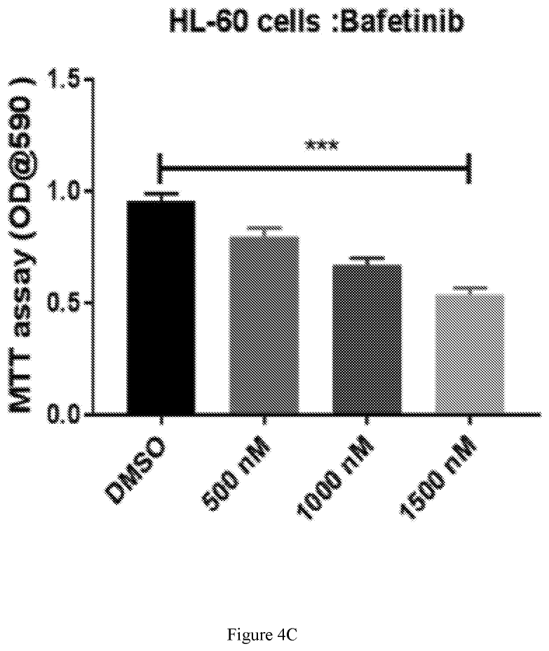

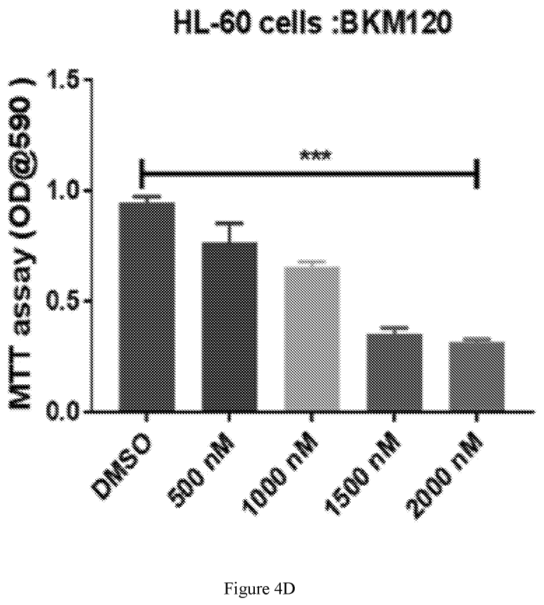

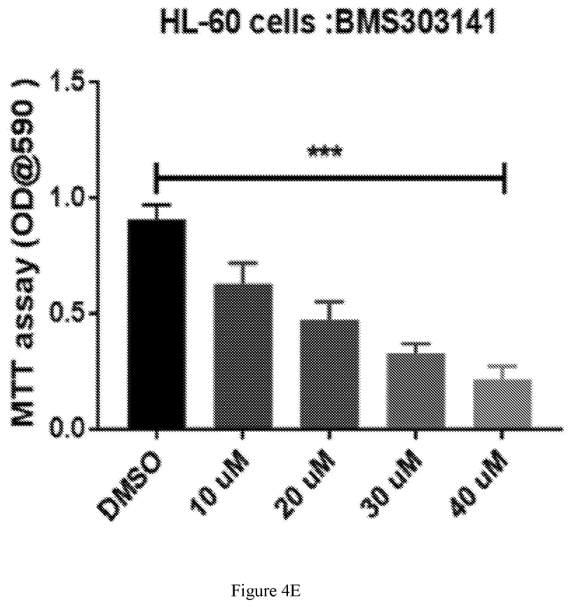

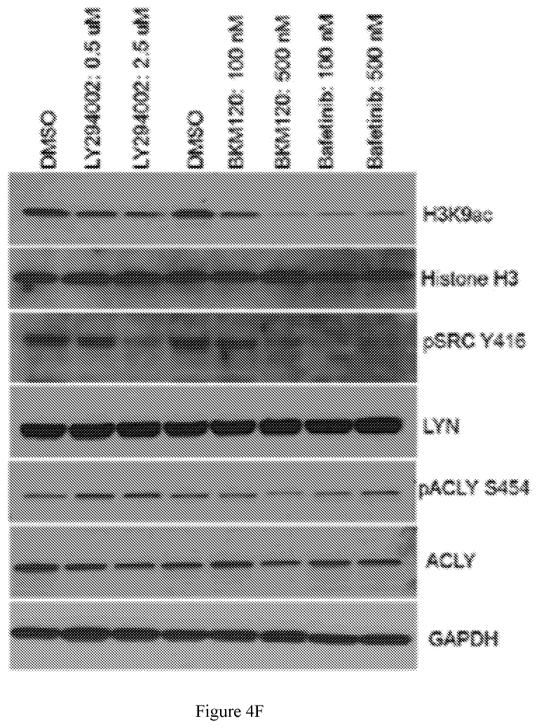

ACLY-mediated production of Acetyl-CoA is sensitive to Lyn tyrosine kinase inhibitor in AML (FIGS. 4A-4C). To determine whether Lyn plays a role in ACLY activation, kidney embryonic HEK293T cells were transfected either with HA-tagged ACLY alone or with HA-tagged ACLY and Src. FIG. 3A shows that the 120-kDa strongly tyrosine phosphorylated ACLY protein could be specifically precipitated with HA-conjugated agarose and that this phosphorylation only took place in cells co-transfected with Src. This observation was confirmed by in vitro tyrosine kinase assay on purified ACLY protein and Lyn immunoprecipitates from HL-60 AML cells. In the presence of active pY396-Lyn the ACLY was tyrosine phosphorylated and this process was sensitive to Lyn tyrosine kinase inhibitor (FIG. 3B). These findings show that SFK-dependent pathway, Lyn in AML cells, induces the ACLY activity in protein tyrosine kinase-dependent manner.

### Example 5: Identification of the Tyrosine Residues of ACLY that are Phosphorylated by Lyn and/or Src

Whether any of the tyrosine residues of ACLY could be directly phosphorylated by Src family kinases Lyn or Src was examined. In vitro tyrosine kinase assays were performed on bacterially expressed and purified recombinant full length ACLY protein in the presence of active recombinant Lyn or Src and determined that active recombinant Lyn or Src directly phosphorylated purified ACLY at tyrosine residues (FIG. 3C). The phosphoproteomics analysis of ACLY in vitro phosphorylated samples indicated that Lyn and Src directly phosphorylated ACLY on six and four tyrosine residues, respectively (FIG. 3D). The three ACLY tyrosine residues, Y682, Y252, Y227, were common for Lyn and Src and were located in the catalytic domain, the citrate-binding domain and the ATP-binding domain, respectively (FIG. 3D right panel, FIG. 6A, and FIG. 10).

### Example 6: ACLY Enzyme Activity and Acetyl-CoA Production are Inhibited by PI3K and LYN Inhibitors in AML Cells

To determine whether PI3K and Lyn activity could affect ACLY-mediated synthesis of Acetyl-CoA in AML, HL-60 cells were treated for 16 hours with the specific Lyn inhibitor (Bafetinib) or two structurally and mechanistically distinct inhibitors of PI3K (LY294002 or BKM120), and then ACLY enzyme activity and acetyl-CoA levels was measured. As shown in FIG. 4B, each of the three inhibitors significantly prevented the synthesis of Acetyl-CoA in AML cells. The corresponding control experiments indicated statistically significant inhibition of ACLY enzyme activity in these HL-60 cell lysates (FIG. 4A). Coupled with the fact that PIP₂ and PIP₃ are directly associated with Lyn-phosphorylated ACLY and ACLY is a major enzyme for Acetyl-CoA synthesis, these findings strongly indicate that over-activated PI3K and Lyn in leukemia cells stimulate the ACLY-mediated Acetyl-CoA production.

### Example 7: Growth of AML Cells is Strongly Suppressed by Lyn, PI3K and ACLY Inhibition

ACLY/Acetyl-CoA provides pro-growth and pro-survival signals to the cells, by providing acetyl groups that are required for histone acetylation at growth genes and fatty acids in phospholipid synthesis. In the present study, it was confirmed that the ACLY inhibitor BMS303141 inhibited within 72 hours growth of HL-60 AML cells with an IC50 of ˜10-20 (FIG. 4E). This was lower than the effective doses reported in literature for ACLY-associated growth inhibition in other cells. The similar pattern of growth inhibition within 72 hours was observed with the Lyn inhibitor and PI3K inhibitor (FIG. 4 C and FIG. 4D). Thus, prolonged inhibition of Lyn, PI3K and ACLY can profoundly suppress AML cell growth. These results show that Lyn/PI3K and ACLY/Acetyl-CoA provides pro-proliferation and pro-survival signals in AML cells.

### Example 8: H3K9 Acetylation is Prevented by PI3K and LYN Inhibitors in AML Cells

ACLY/Acetyl-CoA is required for histone acetylation by providing acetyl groups and initiates cell growth by promoting acetylation of histones specifically at growth genes. The active oncogenic N-RAS and other oncogenes, that are expressed in the patient-derived primary AML cells and HL-60 AML cell line, can increase H3K9ac. Acetylation of H3K9 is particularly important, since it is present almost exclusively at growth genes and is highly correlated with active promoters of oncogenes. Since it was observed that the PI3K and Lyn inhibitors prevented ACLY-mediated production of Acetyl-CoA (FIG. 4), it was examined whether these inhibitors could also suppress acetylation of H3K9 in AML cells. Indeed, FIG. 4F shows that both Lyn tyrosine kinase and PI3K inhibitors almost totally blocked H3K9 acetylation in AML cells. These data (together with data in FIGS. 4A-4E) indicate that over-activated PI3K and Lyn in leukemia cells increase histone acetylation and gene activation through stimulating the synthesis of Acetyl-CoA.

### Example 9: Phosphoinositide Fatty Acid Composition is Altered by PI3K and Lyn Inhibitors in AML Cells in a Manner Consistent with ACLY Inhibition

The production of fatty acids/phospholipids requires ACLY/Acetyl-CoA. Since it was found that ACLY enzyme activity and production of Acetyl-CoA were blocked by PI3K and Lyn inhibitors (FIGS. 4A and 4B), and PIP₂/PIP₃/Lyn were directly associated with ACLY, mass spectrometric analysis was used to examine whether these inhibitors affected the fatty acid moieties of phosphoinositides in HL60 AML cells. Inhibitors suppressed PI, PIP and PIP₂ formation, especially saturated and monounsaturated species with shorter fatty acid chains (FIG. 5 and FIG. 9). Specifically, 32:0, 34:0 and 36:0 PI, PIP and PIP₂ decreased most dramatically, according to the following order (32:0>34:0>36:0; PI>PIP>PIP₂) (FIG. 5 and FIG. 9). This differential inhibition is consistent with ACLY/Acetyl-CoA inhibition since ACLY activity generates shorter chain fatty acids first which are the precursors for longer chain fatty acids. Thus, the inhibition remodeled the overall phosphoinositide fatty acid profile and reduced total levels of phosphoinositides. Both mechanistically distinct inhibitors of PI3K and the Lyn inhibitor dramatically reduced PI/PIP/PIP₂ synthesis in leukemia cells (FIG. 9). These findings indicate that over-activated PI3K and Lyn in leukemia cells augment phosphoinositide synthesis (including PIP₂) through activation of ACLY/Acetyl-CoA.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A pharmaceutical composition comprising:
a Src protein tyrosine kinase inhibitor;
an ATP citrate lyase (ACLY) inhibitor;
a PI3K inhibitor; and
a pharmaceutically acceptable carrier.

2. The pharmaceutical composition according to claim 1, wherein the ACLY inhibitor is BMS303141, MEDICA16, SB204990, or NDI-091143.

3. The pharmaceutical composition according to claim 1 or claim 2, wherein the ACLY inhibitor is present in an amount from about 1 mg to about 500 mg, from about 50 mg to about 400 mg, from about 75 mg to about 300 mg, or from about 100 mg to about 200 mg.

4. The pharmaceutical composition according to any one of claims 1 to 3, wherein the PI3K inhibitor is LY294002, BKM120, voxtalisib, umbralisib, copanlisib, duvelisib, or alpelisib.

5. The pharmaceutical composition according to any one of claims 1 to 4, wherein the PI3K inhibitor is present in an amount from about 1 mg to about 500 mg, from about 50 mg to about 400 mg, from about 75 mg to about 300 mg, or from about 100 mg to about 200 mg.

6. The pharmaceutical composition according to any one of claims 1 to 5, wherein the Src protein tyrosine kinase inhibitor is a Lyn tyrosine kinase inhibitor.

7. The pharmaceutical composition according to claim 6, wherein the Lyn tyrosine kinase inhibitor is bafetinib, bosutinib, masitinib, soracatinib, AZ 628, TC-S 7003, or PRT 062607.

8. The pharmaceutical composition according to claim 6 or claim 7, wherein the Lyn tyrosine kinase inhibitor is present in amount from about 1 mg to about 100 mg, from about 5 mg to about 75 mg, from about 10 mg to about 60 mg, or from about 12.5 mg to about 50 mg.

9. The pharmaceutical composition according to any one of claims 1 to 8, wherein the pharmaceutical composition is an oral dosage formulation, an intravenous dosage formulation, a topical dosage formulation, an intraperitoneal dosage formulation, or an intrathecal dosage formulation.

10. The pharmaceutical composition according to any one of claims 1 to 9, wherein the oral dosage formulation is a pill, tablet, capsule, cachet, gel-cap, pellet, powder, granule, or liquid.

11. The pharmaceutical composition according to any one of claims 1 to 10, wherein the oral dosage formulation is protected from light and present within a blister pack or bottle, or the intravenous dosage formulation is present within an intravenous bag.

12. The pharmaceutical composition according to any one of claims 9 to 11, wherein the oral dosage formulation is a capsule.

13. The pharmaceutical composition according to claim 12, wherein the capsule comprises about 12.5 mg, about 25 mg, about 37.5 mg, or about 50 mg of the Lyn tyrosine kinase inhibitor.

14. The pharmaceutical composition according to claim 12 or claim 13, wherein the capsule comprises about 25 mg, about 50 mg, about 75 mg, about 100 mg, about 150 mg, or about 200 mg of the ACLY inhibitor.

15. The pharmaceutical composition according to any one of claims 12 to 14, wherein the capsule comprises about 25 mg, about 50 mg, about 75 mg, about 100 mg, about 150 mg, or about 200 mg of the PI3K inhibitor.

16. A method of identifying a compound as a potential therapeutic agent for treating a disease or condition associated with the ACLY/Acetyl-CoA metabolic pathway in a cell comprising:
performing an assay to determine the ability of the compound to inhibit the interaction of PIP₂, PIP₃, and/or Lyn tyrosine kinase to ACLY, or the activity of a complex of PIP₂/Lyn tyrosine kinase/ACLY, or the activity of complex of PIP₃/Lyn tyrosine kinase/ACLY;
wherein when the compound inhibits the interaction of PIP₂, PIP₃, and/or Lyn tyrosine kinase to ACLY, or inhibits the activity a complex of PIP₂/Lyn tyrosine kinase/ACLY, or inhibits the activity of a complex of PIP₃/Lyn tyrosine kinase/ACLY, the compound is a potential therapeutic agent.

17. The method according to claim 16, wherein the compound is a small molecule.

18. The method according to claim 16 or claim 17, wherein the disease or condition associated with the ACLY/Acetyl-CoA metabolic pathway is a cancer, high cholesterol, inflammation, atherosclerotic cardiovascular disease (ASCVD), nonalcoholic fatty liver disease (NAFLD), or cancer-associated fibrosis.

19. The method according to claim 18, wherein the cancer is acute myeloid leukemia (AML), chronic myeloid leukemia (CML), chronic lymphocytic leukemia (CLL), acute lymphoblastic leukemia (ALL), lymphoma, breast cancer, pancreatic cancer, glioblastoma, or prostate cancer.

20. The method according to any one of claims 16 to 19, wherein the assay is in silico computational modeling.

21. The method according to any one of claims 16 to 19, wherein the assay is a binding assay, an ACLY enzymatic activity assay, an ACLY phosphorylation assay, an ACLY-mediated acetyl-CoA assay, an ACLY/acetyl-CoA-mediated histone acetylation assay, or an ACYL/acetyl-CoA-mediated fatty acid and lipid synthesis assay.

22. The method according to claim 21, wherein the binding assay is a high throughput binding assay.

23. The method according to any one of claims 16 to 22, wherein the compound inhibits the interaction of PIP₂ and/or PIP₃ to ACLY.

24. The method according to any one of claims 16 to 22, wherein the compound inhibits the interaction of Lyn tyrosine kinase to ACLY.

25. The method according to any one of claims 16 to 22, wherein the compound inhibits the interaction of both PIP₂ and Lyn tyrosine kinase to ACLY.

26. The method according to any one of claims 16 to 22, wherein the compound inhibits the activity a complex of PIP₂/Lyn tyrosine kinase/ACLY.

27. The method according to any one of claims 16 to 22, wherein the compound inhibits the activity a complex of PIP₃/Lyn tyrosine kinase/ACLY.

28. A method of treating a disease or condition associated with the ACLY/Acetyl-CoA metabolic pathway in a cell in a subject in need thereof comprising administering to the subject a Lyn tyrosine kinase inhibitor, an ACLY inhibitor, and a PI3K inhibitor to the subject.

29. The method according to claim 28, wherein the disease or condition associated with the ACLY/Acetyl-CoA metabolic pathway is a cancer, high cholesterol, inflammation, atherosclerotic cardiovascular disease (ASCVD), nonalcoholic fatty liver disease (NAFLD), or cancer-associated fibrosis.

30. The method according to claim 29, wherein the cancer is acute myeloid leukemia (AML).

31. The method according to any one of claims 28 to 30, wherein the Lyn tyrosine kinase inhibitor, the ACLY inhibitor, and the PI3K inhibitor are administered to the subject together in a single pharmaceutical composition.

32. A combination of a Lyn tyrosine kinase inhibitor, an ACLY inhibitor, and a PI3K inhibitor for use in the manufacture of a medicament for treating a disease or condition associated with the ACLY/Acetyl-CoA metabolic pathway in a cell.

33. Use of a pharmaceutical composition comprising a Lyn tyrosine kinase inhibitor, an ACLY inhibitor, and a PI3K inhibitor for treating a disease or condition associated with the ACLY/Acetyl-CoA metabolic pathway in a cell.
